(19) United States
(12) Patent Application Publication
(10) Pub. No.: US 2025/0382461 A1
(43) Pub. Date: Dec. 18, 2025

(54) POLYROTAXANE COMPRISING CYCLIC MOLECULE WITH ALDEHYDE GROUP ADDUCT, METHOD FOR PRODUCING SAID POLYROTAXANE, STRETCHABLE BIOMATERIAL, AND METHOD FOR PRODUCING SAID BIOMATERIAL

(71) Applicant: KOKEN CO., LTD., Tokyo (JP)

(72) Inventor: Riku KUBOTA, Yamagata (JP)

(73) Assignee: KOKEN CO., LTD., Tokyo (JP)

(21) Appl. No.: 19/103,849

(22) PCT Filed: Aug. 4, 2023
(86) PCT No.: PCT/JP2023/028657

§ 371 Date: Feb. 14, 2025

(30) Foreign Application Priority Data
Aug. 16, 2022 (JP) 2022-129675

(51) Int. Cl. C08L 89/00; C08L 3/02

(52) U.S. Cl. CPC C08L 89/00; C08L 3/02

(57) ABSTRACT

To develop a biomaterial having stretchability (in particular, collagen having stretchability), the inventors of the present invention have found a method of producing an aldehyde group-added cyclic molecule-containing polyrotaxane by which an aldehyde group can be specifically added to a cyclic molecule of a polyrotaxane, and a method of producing a biomaterial having stretchability, the method including reductive amination and a crosslinking method by which the production of a free aldehyde can be suppressed. Further, the inventors have recognized that thread-like collagen has stretchability. Thus, the inventors have completed the present disclosure.

### TECHNICAL FIELD

The present disclosure relates to an aldehyde group-added cyclic molecule-containing polyrotaxane and a method of producing the polyrotaxane, and a biomaterial having stretchability and a method of producing the biomaterial.

The present application claims the priority of Japanese Patent Application 2022-129675 incorporated herein by reference.

### (Macrocyclic Compound)

A macrocyclic compound is useful in the development of a functional material in the field of materials chemistry. In particular, a cyclodextrin (α-, β-, or γ-CD) has been attracting large attention because the cyclodextrin has such features as described below (Non Patent Literature 1): the cyclodextrin has high water solubility; its mass production is easy; and the cyclodextrin is suitable for chemical modification. In addition, a cavity inside the CD can encapsulate the amphiphilic moiety or hydrophobic moiety of a molecule through an inclusion phenomenon, and hence the CD has large potential as an inclusion compound that may be used in, for example, a drug delivery system, an artificial enzyme, or a chemical sensor (Non Patent Literature 2).

### (Polyrotaxane)

A polyrotaxane (PR) formed of the CD and an amphiphilic polymer, such as polyethylene glycol (PEG) or polypropylene glycol (PPG), is a promising soft material. The PEG or the PPG penetrates into the cavity of the CD from both the primary hydroxy group side and secondary hydroxy group side thereof. As a result, CD molecules are randomly oriented in the same direction and the opposite direction along a PR chain, and hence can freely move and rotate on the chain. Thus, a composite material having a feature called a slide ring effect is obtained. Such feature leads to improvements in mechanical properties, and unique properties, such as stimulus responsiveness and a self-healing property (Non Patent Literature 3).

### (Collagen)

Collagen is a highly biocompatible protein for forming a large part of the organs and tissues of mammals. Further, atelocollagen that is free of telopeptides at its N-terminal and C-terminal shows relatively low immunogenicity as compared to that of the collagen. Each of the collagen and the atelocollagen has currently been viewed as promising for the achievement of a human-friendly biomaterial that can be used both in vivo and in vitro. More specifically, the atelocollagen has been developed as a biomaterial applicable to, for example, nucleic acid delivery, regenerative medicine, or drug discovery.

The collagen and the atelocollagen can each be processed into various shapes (e.g., gel, sponge, a filament, a membrane, and a fiber), and hence can be used in basic research and clinical research.

### (Thread-Like Collagen)

A plurality of kinds of thread-like collagen have been reported (Patent Literatures 1 to 3).

However, thread-like collagen crosslinked with a polyrotaxane has not been disclosed.

### CITATION LIST

### Patent Literature

[PTL 1] JP 2005-314865 A
[PTL 2] JP 2003-193328 A
[PTL 3] JP 2017-086066 A

### Non Patent Literature

[NPL 1] Chem. Soc. Rev. 2017, 46 (9), 2459-2478.
[NPL 2] Chem. Soc. Rev. 2005, 34 (2), 120-132.
[NPL 3] Chem. Commun. 2020, 56 (32), 4381-4395.

### SUMMARY OF INVENTION

### Technical Problem

The inventors of the present invention have aimed to develop a biomaterial having stretchability (in particular, collagen having stretchability). In view of the foregoing, the inventors of the present invention have attempted to produce thread-like collagen crosslinked with a polyrotaxane.

However, the inventors have recognized that (1) it is difficult to crosslink the polyrotaxane to a biomaterial by a related-art method. Further, the inventors have recognized that (2) a functional group (imine, also referred to as “Schiff base”) formed by the crosslinking may cause hydrolysis in a living organism to produce a free aldehyde.

### Solution to Problem

To cope with the above-mentioned problem (1), the inventors of the present invention have found a method of producing an aldehyde group-added cyclic molecule-containing polyrotaxane by which an aldehyde group can be specifically added to a cyclic molecule of a polyrotaxane. In addition, to cope with the above-mentioned problem (2), the inventors have found a method of producing a biomaterial having stretchability, the method including reductive amination and a crosslinking method by which the production of a free aldehyde can be suppressed.

Further, the inventors have recognized that thread-like collagen has stretchability. Thus, the inventors have completed the present disclosure.

In addition, the inventors of the present invention have recognized that thread-like collagen that has been made tougher is obtained by controlling the inclusion ratio of the cyclic molecules of an aldehyde group-added cyclic molecule-containing polyrotaxane. Thus, the inventors have further completed the present disclosure.

The present disclosure is as described below.

1. An aldehyde group-added cyclic molecule-containing polyrotaxane, including the following:
a linear molecule;
capping groups (stopper molecules), provided that the capping groups are positioned at both terminals of the linear molecule; and an aldehyde group-added cyclic molecule, provided that an inside of the aldehyde group-added cyclic molecule is penetrated by the linear molecule.

2. The polyrotaxane according to the above-mentioned item 1, wherein the polyrotaxane is a water-soluble polyrotaxane.

3. The polyrotaxane according to the above-mentioned item 1 or 2, wherein the aldehyde group-added cyclic molecule is substantially free of a ketone group added thereto.

4. A crosslinking composition, including the polyrotaxane of the above-mentioned item 1 or 2.

5. The crosslinking composition according to the above-mentioned item 4, wherein the crosslinking composition is used for crosslinking a biomaterial.

6. The polyrotaxane according to the above-mentioned item 1 or 2, wherein the linear molecule is a constituent unit based on polyethylene glycol, and the aldehyde group-added cyclic molecule is a constituent unit based on an aldehyde group-added cyclodextrin.

7. The polyrotaxane according to the above-mentioned item 1 or 2, wherein the linear molecule is a constituent unit based on polypropylene glycol, and the aldehyde group-added cyclic molecule is a constituent unit based on an aldehyde group-added cyclodextrin.

8. The polyrotaxane according to the above-mentioned item 1 or 2, wherein the linear molecule is a constituent unit based on poly(ethylene glycol)-block-poly(propylene glycol)-block-poly(ethylene glycol), and the aldehyde group-added cyclic molecule is a constituent unit based on an aldehyde group-added cyclodextrin.

9. A method of producing an aldehyde group-added cyclic molecule-containing polyrotaxane, the method including the following steps:
(1) a step of bringing a linear molecule and a cyclic molecule having a hydroxyl group into contact with each other to provide a compound 3 in which an inside of the cyclic molecule having a hydroxyl group is penetrated by the linear molecule;
(2) a step of bringing capping groups and the compound 3 into contact with each other to provide a compound 2 in which the inside of the cyclic molecule having a hydroxyl group is penetrated by the linear molecule having the capping groups at both terminals thereof; and
(3) a step of subjecting the compound 2 to oxidation treatment in the presence of a TEMPO derivative and an iodobenzene derivative to provide the aldehyde group-added cyclic molecule-containing polyrotaxane.

10. The production method according to the above-mentioned item 9, wherein in the step (2), the compound 3 is dissolved in tetrahydrofuran.

11. The production method according to the above-mentioned item 9 or 10, wherein in the step (3), N,N-diisopropylethylamine is further caused to be present.

12. The production method according to the above-mentioned item 9 or 10, wherein in the step (3), the compound 2 is dissolved in hexamethylphosphoric triamide, N,N-dimethylformamide, or dimethyl sulfoxide.

13. The production method according to the above-mentioned item 9 or 10, wherein in the step (3), the compound 2 is dissolved in hexamethylphosphoric triamide containing N,N-diisopropylethylamine.

14. The production method according to the above-mentioned item 9 or 10, wherein the linear molecule and the cyclic molecule having a hydroxyl group are selected so that an inclusion ratio, which is a molar fraction of aldehyde group-added cyclic molecules per number of repeating units in the linear molecule, becomes from 1 mol % to 40 mol %.

15. The production method according to the above-mentioned item 9 or 10, wherein the inclusion ratio is from 2 mol % to 15 mol %.

16. A biomaterial subjected to crosslinking treatment with a polyrotaxane, the polyrotaxane including the following:
a linear molecule;
capping groups (stopper molecules), provided that the capping groups are positioned at both terminals of the linear molecule; and
a cyclic molecule, provided that an inside of the cyclic molecule is penetrated by the linear molecule.

17. The biomaterial according to the above-mentioned item 16, wherein the polyrotaxane is an aldehyde group-added cyclic molecule-containing polyrotaxane.

18. The biomaterial according to the above-mentioned item 17, wherein the aldehyde group-added cyclic molecule is substantially free of a ketone group added thereto.

19. The biomaterial according to the above-mentioned item 17 or 18, wherein the biomaterial is collagen.

20. The biomaterial according to the above-mentioned item 17 or 18, wherein the biomaterial is thread-like collagen.

21. The biomaterial according to the above-mentioned item 20, wherein the linear molecule is a constituent unit based on polyethylene glycol, polypropylene glycol, or poly(ethylene glycol)-block-poly(propylene glycol)-block-poly(ethylene glycol), the aldehyde group-added cyclic molecule is a constituent unit based on an aldehyde group-added cyclodextrin, and the biomaterial is thread-like collagen having stretchability.

22. The biomaterial according to the above-mentioned item 21, wherein the biomaterial has the following properties:
(1) a fracture stress of from 280 kPa to 3,200 kPa;
(2) a fracture strain of from 40% to 70%;
(3) an elastic modulus of from 18 kPa to 220 kPa; and
(4) a toughness of from 83 kJ/m³ to 350 kJ/m³.

23. The biomaterial according to the above-mentioned item 22, wherein the biomaterial further has the following property:
(1) a stress of from 10 kPa to 1,000 kPa at a time of loading of a strain of from 30% to 40%.

24. A method of producing a biomaterial subjected to crosslinking treatment with a polyrotaxane, the method including the following step:
(1) a step of subjecting a biomaterial having a lysine residue to reductive amination reaction treatment in the presence of an aldehyde group-added cyclic molecule-containing polyrotaxane.

25. The production method according to the above-mentioned item 24, wherein the step (1) is a step of bringing the biomaterial having a lysine residue into contact with a buffer containing the aldehyde group-added cyclic molecule-containing polyrotaxane to subject the biomaterial having a lysine residue to the reductive amination reaction treatment.

26. The production method according to the above-mentioned item 25, wherein the buffer in the step (1) contains a hydride reducing agent.

### Advantageous Effects of Invention

The present disclosure has one or more of the following effects:
(1) the provision of the aldehyde group-added cyclic molecule-containing polyrotaxane (in particular, the aldehyde group-added cyclic molecule-containing polyrotaxane in which a cyclic molecule is substantially free of a ketone group added thereto);
(2) the provision of the water-soluble aldehyde group-added cyclic molecule-containing polyrotaxane;
(3) the provision of the method of producing an aldehyde group-added cyclic molecule-containing polyrotaxane by which an aldehyde group can be specifically added to a cyclic molecule of a polyrotaxane;
(4) the provision of the method of producing a biomaterial having stretchability, the method including a crosslinking method by which the production of a free aldehyde can be suppressed;
(5) the provision of the biomaterial having stretchability (in particular, the collagen having stretchability); and
(6) the provision of the aldehyde group-added cyclic molecule-containing polyrotaxane in which the inclusion ratio of its cyclic molecules is controlled, and the production method therefor.

### BRIEF DESCRIPTION OF DRAWINGS

FIG. 1 is a synthesis scheme 1 for an aldehyde group-added cyclic molecule-containing polyrotaxane of the present disclosure.

FIG. 2 is a schematic view of the production of thread-like atelocollagen of the present disclosure. In a tensile test, the thread-like atelocollagen of the present disclosure was fixed to a plastic sheet measuring 2.5 cm by 4.5 cm.

FIG. 3 are the one-dimensional and two-dimensional NMR spectra of the aldehyde group-added cyclic molecule-containing polyrotaxane of the present disclosure (500 MHz, D₂O, 298 K) (R=H, D). FIG. 3a is a ¹H NMR spectrum and FIG. 3b is a NOESY spectrum. Measurement was performed in D₂O containing 15 mM of a PRβCD1 (deuteration ratio: 100%). A relaxation time was set to 2 seconds, and a mixing time was set to 0.68 second.

FIG. 4 are graphs showing the mechanical properties of produced thread-like atelocollagen (Col-PRβCD1 or Col-PRαCD1). FIG. 4a is a typical stress-strain curve of Col alone, the Col-PRβCD1, the Col-PRαCD1, or Col-glutaraldehyde (GA), FIG. 4b is a graph showing the fracture stress thereof, FIG. 4c is a graph showing the fracture strain thereof, FIG. 4d is a graph showing the Young's modulus thereof, and FIG. 4e is a graph showing the toughness thereof. The fracture stress was calculated by dividing a fracture test force (N) by the sectional area (about 0.2 mm²) of a thread. The fracture strain was calculated as a ratio between the length of elongation of a thread sample at the time of its fracture and the initial length (25 mm) thereof. The Young's modulus was determined in the initial region of the stress-strain curve in which the stress and the strain linearly correlated with each other. The toughness was calculated as the area of the stress-strain curve. Experimental data was represented by a mean±S.D. (n=6). Tukey's test was used in statistical analysis (*p<0.05, **p<0.01). The abbreviation “n.s.” means that no significant difference is present. The abbreviation “Col-GA” means an atelocollagen-GA thread (two crosslinking sites are present in glutaraldehyde, and GA is bonded to the collagen at each of the crosslinking sites), the abbreviation “Col-PRβCD1” means a PRβCD1-crosslinked atelocollagen thread, the abbreviation “Col-PRαCD1” means a PRαCD1-crosslinked atelocollagen thread, and the abbreviation “Col alone” means an atelocollagen thread free of GA, the PRβCD1, or the PRαCD1.

FIG. 5 is a graph showing a stress when a strain load is repeatedly applied to the Col-PRβCD1 thread. During 50 times of repeated measurement, the thread elongated in the relative strain range of from 30% to 40%.

FIG. 6 is a synthesis scheme 2 for the aldehyde group-added cyclic molecule-containing polyrotaxane of the present disclosure.

FIG. 7 is the ¹H NMR spectrum of a PRαCD3 (DMSO-d₆, 400 MHz, 298 K).

FIG. 8 is the ¹H NMR spectrum of a PRαCD2 (DMSO-d₆, 400 MHz, 298 K).

FIG. 9 is the ¹H NMR spectrum of the PRαCD1 (DMSO-d₆/D₂O, 400 MHz, 298 K).

FIG. 10 is a schematic view of stretchable thread-like collagen crosslinked with the aldehyde group-added cyclic molecule-containing polyrotaxane.

FIG. 11 is the ¹H NMR spectrum of PCD (D₂O, 400 MHz, 298 K).

FIG. 12 is the ¹H NMR spectrum of PCD after oxidation treatment with DMP (D₂O, 400 MHz, 298 K).

FIG. 13 is the ¹H NMR spectrum of PCD after oxidation treatment with a TEMPO/PhI(OAc)₂ redox couple (D₂0, 400 MHz, 298 K).

FIG. 14 is the ¹H NMR spectrum of a PluPRβCD3 (CDCl₃, 400 MHz, 293 K). The PluPRβCD3 includes polyethylene glycol (x=85) and polypropylene glycol (y=30).

FIG. 15 is the ¹H NMR spectrum of a PluPRβCD2 (DMSO-d₆, 400 MHz, 293 K). The PluPRβCD2 includes polyethylene glycol (x=85) and polypropylene glycol (y=30).

FIG. 16a and FIG. 16b are the ¹H NMR spectra of a PegPRαCD1-DMHZ and a PluPRβCD1-DMHZ, respectively (DMSO-d₆, 400 MHz, 318 K).

FIG. 17 are a photograph and graphs showing the evaluations of mechanical properties by a tensile test. FIG. 17a is a photograph during the performance of the tensile test, FIG. 17b is a typical stress-strain curve, FIG. 17c is a graph showing a fracture stress, FIG. 17d is a graph showing a toughness, FIG. 17e is a graph showing a Young's modulus, and FIG. 17f is a graph showing a fracture elongation percentage. The fracture stress was calculated by dividing a test force at the time of fracture by a sectional area. The Young's modulus was calculated as the slope of the initial linear region (at an elongation percentage of up to 6%) of the stress-strain curve. Tukey's test was used in statistical analysis (*p<0.01, **p<0.05).

### DESCRIPTION OF EMBODIMENTS

### (Targets of the Present Disclosure)

The targets of the present disclosure are an aldehyde group-added cyclic molecule-containing polyrotaxane (in particular, an aldehyde group-added cyclic molecule-containing polyrotaxane in which a cyclic molecule is substantially free of a ketone group added thereto), a method of producing an aldehyde group-added cyclic molecule-containing polyrotaxane by which an aldehyde group can be specifically added to a cyclic molecule of a polyrotaxane (hereinafter sometimes abbreviated as “method of producing an aldehyde group-added cyclic molecule-containing polyrotaxane of the present disclosure”), a biomaterial having stretchability (in particular, collagen having stretchability), a method of producing a biomaterial having stretchability, the method including a crosslinking method by which the production of a free aldehyde can be suppressed (hereinafter sometimes abbreviated as “method of producing a biomaterial having stretchability of the present disclosure”), and an aldehyde group-added cyclic molecule-containing polyrotaxane in which the inclusion ratio of its cyclic molecules is controlled and a production method therefor.

### (Aldehyde Group-Added Cyclic Molecule-Containing Polyrotaxane)

The aldehyde group-added cyclic molecule-containing polyrotaxane of the present disclosure includes the following:
(1) a linear molecule;
(2) capping groups (stopper molecules), provided that the capping groups are positioned at both the terminals of the linear molecule; and
(3) an aldehyde group-added cyclic molecule, provided that the inside of the aldehyde group-added cyclic molecule is penetrated by the linear molecule.

Examples below have recognized that the aldehyde group-added cyclic molecule-containing polyrotaxane of the present disclosure has water solubility. More specifically, the presence of one aldehyde group or one or more aldehyde groups in one cyclic molecule may make the polyrotaxane water-soluble.

In addition, an aldehyde group-added cyclic molecule-containing polyrotaxane obtained by the method of producing an aldehyde group-added cyclic molecule-containing polyrotaxane of the present disclosure includes an aldehyde group-added cyclic molecule that is substantially free of a ketone group added thereto.

The phrase “substantially free of a ketone group added thereto” not only means that the cyclic molecule is completely free of a ketone group added thereto but also includes a case in which the number of ketone groups to be added is so small that there are no influences by the ketone groups. More specifically, according to the results of Examples below, the phrase means that 95% or more, 96% or more, 97% or more, 98% or more, or 99% or more of the hydroxy groups of the cyclic molecule are substituted with ketone groups.

### (Linear Molecule)

The linear molecule is not limited as long as the molecule is a known linear molecule used in a polyrotaxane. However, it is preferred that no aldehyde group, hydroxyl group, or amino group be added to or liberated from the molecule, or the molecule be free of such group. However, a linear molecule having such group may be used as long as such group is protected with a certain protective group. In addition, the linear molecule may include a branched chain.

The linear molecule may be, for example, a constituent unit based on polyethylene glycol (PEG), polypropylene glycol (PPG), a polyethylene glycol-polypropylene glycol copolymer, poly(ethylene glycol)-block-poly(propylene glycol)-block-poly(ethylene glycol), PES15 polyester polyol, a viologen polymer, linear polyethylene imine, Ionene-6.10, a polylactic acid-polyethylene glycol-polylactic acid triblock copolymer, a polylactic acid-polyethylene glycol block copolymer, or polydimethylsiloxane.

### (Capping Groups)

The capping groups (stopper molecules) are not limited as long as the groups are each a structural body having not less than a size that can prevent the aldehyde group-added cyclic molecule from detaching from the linear molecule, and the groups are known capping groups used in a polyrotaxane. However, it is preferred that no amino group be added to or liberated from each of the capping groups, or the capping group be free of an amino group. However, a capping group having such group may be used as long as such group is protected with a certain protective group. In addition, an aldehyde group may be added to a capping group using a cyclodextrin.

An example thereof may be a constituent unit based on a triazine derivative represented by the following general formula (I), α-cyclodextrin, β-cyclodextrin, γ-cyclodextrin, adamantane, o,m-dinitrobenzene, tritylglycine, adamantanecarboxylic acid, tritylamide, fluorescein isothiocyanate, tritylaniline, tritylphenol, or trityl chloride.

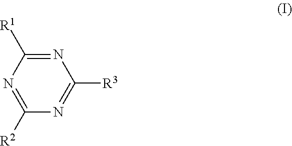

In the formula, R¹ to R³ may be identical to or different from each other, and each represent a hydrogen atom, a hydroxymethylamino group, an amino group, a hydroxy group, a halogen atom, an aryl group, a linear or branched alkyl group or alkenyl group having 1 or more and 6 or less carbon atoms, 4-(aminomethyl)-N-methylaniline, or a linear or branched alkoxy group or alkenyloxy group having 1 or more and 6 or less carbon atoms.

Specific examples thereof may include N2,N4-bis(4-(aminomethyl)phenyl)-6-chloro-1,3,5-triazine-2,4-diamine, 2,4-diamino-1,3,5-triazine, 2-chloro-4,6-diamino-1,3,5-triazine, 2,4,6-triamino-1,3,5-triazine, 2,4,6-trihydroxy-1,3,5-triazine, and trichloro-1,3,5-triazine.

A preferred capping group (stopper molecule) is represented by the following formula (1). Amino groups may each be protected with a protective group or the like as required.

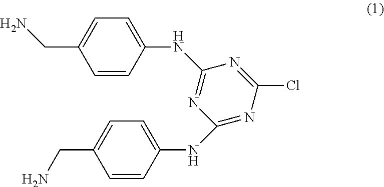 [SMILES: NCc1ccc(Nc2nc(Cl)nc(Nc3ccc(CN)cc3)n2)cc1]

### (Cyclic Molecule)

The cyclic molecule is not limited as long as the molecule is a known cyclic molecule used in a polyrotaxane. However, a OH group needs to be present in the molecule.

Examples thereof may include constituent units based on α-cyclodextrin, β-cyclodextrin, γ-cyclodextrin, cycloawaodorin, 2-hydroxymethyl-12-crown-4, 2-hydroxymethyl-15-crown-5, and 2-hydroxymethyl-18-crown-6.

A functional group may be added to the cyclic molecule as long as the group is protected with a protective group.

(Aspect of Aldehyde Group-Added Cyclic Molecule-Containing Polyrotaxane) A preferred aspect of the aldehyde group-added cyclic molecule-containing polyrotaxane of the present disclosure is as described below.

The linear molecule is a constituent unit based on polyethylene glycol, polypropylene glycol, or poly(ethylene glycol)-block-poly(propylene glycol)-block-poly(ethylene glycol).

The capping groups (stopper molecules) are each a structural body having not less than a size that can prevent the aldehyde group-added cyclic molecule from detaching from the linear molecule.

The aldehyde group-added cyclic molecule is a constituent unit based on an aldehyde group-added cyclodextrin.

### (Method of Producing Aldehyde Group-Added Cyclic Molecule-Containing Polyrotaxane of the Present Disclosure)

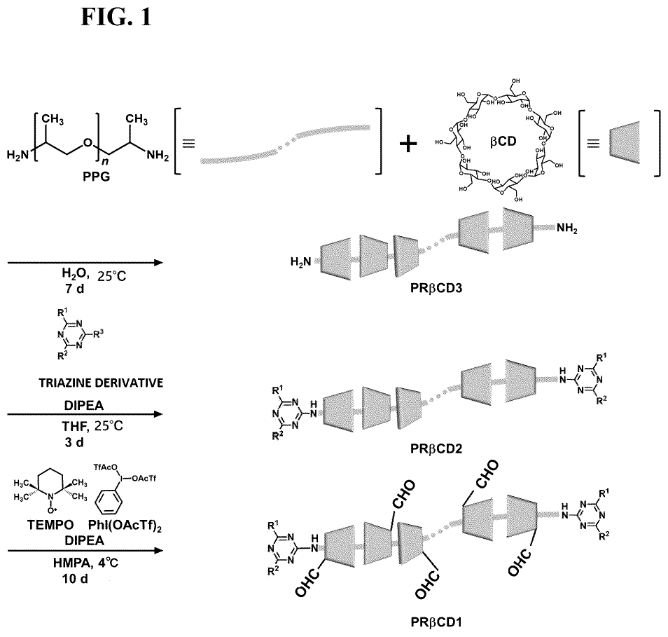

As illustrated in FIG. 1, the method of producing an aldehyde group-added cyclic molecule-containing polyrotaxane of the present disclosure includes the following steps:
(1) a step of bringing a linear molecule and a cyclic molecule having a hydroxyl group into contact with each other to provide a compound 3 in which an inside of the cyclic molecule having a hydroxyl group is penetrated by the linear molecule, provided that stirring is performed as required;
(2) a step of bringing capping groups and the compound 3 into contact with each other to provide a compound 2 in which the inside of the cyclic molecule having a hydroxyl group is penetrated by the linear molecule having the capping groups at both terminals thereof, provided that stirring is performed as required; and
(3) a step of subjecting the compound 2 to oxidation treatment in the presence of a TEMPO derivative and an iodobenzene derivative to provide the aldehyde group-added cyclic molecule-containing polyrotaxane.

Conditions known per se may be adopted as a temperature, a pressure, a solvent, and the like in the above-mentioned steps.

In the step (2), it has been difficult for the inventors of the present invention to produce the target “compound in which the inside of the cyclic molecule having a hydroxyl group is penetrated by the linear molecule having the capping groups at both terminals thereof” under conditions used in a related-art polyrotaxane (an oxidizing agent, a solvent, and capping groups to be used).

In view of the foregoing, in the production method of the present disclosure, unlike a related-art polyrotaxane production method, the triazine derivative represented by the general formula (I) (in particular, the compound represented by the formula (1)), which is a capping group that has not heretofore been known, is preferably used by being dissolved in hexamethylphosphoric triamide and/or tetrahydrofuran serving as a solvent.

With regard to the step (3), in the section “∘Reaction between βCD and DMP” in Examples below, when Dess-Martin periodinane (DMP) and DMSO, which were an oxidizing agent and a solvent to be used in a related-art method, respectively, were used, both the primary and secondary alcohol moieties of a CD were oxidized. Accordingly, the product became a mixture, and was hence not able to be isolated.

Meanwhile, in the section “∘Reaction between βCD and TEMPO/PhI(OAc)₂ Redox Couple” in Examples below, βCD monoaldehyde was able to be obtained as a white solid.

In view of the foregoing, in the production method of the present disclosure, unlike the related-art polyrotaxane production method, the compound in which the inside of the cyclic molecule having a hydroxyl group is penetrated by the linear molecule having the capping groups at both of its terminals is preferably subjected to oxidation treatment in the presence of the TEMPO derivative and the iodobenzene derivative (and in the presence of N,N-diisopropylethylamine as required). Further, the compound is preferably dissolved in hexamethylphosphoric triamide, N,N-dimethylformamide, or dimethyl sulfoxide (more preferably hexamethylphosphoric triamide containing N,N-diisopropylethylamine).

Thus, an aldehyde group can be specifically introduced into a polyrotaxane under a mild condition.

A commercially available product may be used as the TEMPO derivative. Examples thereof may include 4-(2-iodoacetamido)-2,2,6,6-tetramethylpiperidine 1-oxyl free radical, 2,2,6,6-tetramethylpiperidine 1-oxyl free radical, 4-amino-2,2,6,6-tetramethylpiperidine 1-oxyl free radical, 4-oxo-2,2,6,6-tetramethylpiperidine 1-oxyl free radical, 4-oxo-2,2,6,6-tetramethylpiperidine 1-oxyl free radical, 4-hydroxy-2,2,6,6-tetramethylpiperidine 1-oxyl free radical, 4-carboxy-2,2,6,6-tetramethylpiperidine 1-oxyl free radical, 4-methacryloyloxy-2,2,6,6-tetramethylpiperidine 1-oxyl free radical, 4-glycidyloxy-2,2,6,6-tetramethylpiperidine 1-oxyl free radical, 4-isothiocyanato-2,2,6,6-tetramethylpiperidine 1-oxyl free radical, 4-amino-2,2,6,6-tetramethylpiperidine 1-oxyl free radical, 2,2,6,6-tetramethylpiperidine 1-oxyl free radical, 2-hydroxy-2-azaadamantane, 4-hydroxy-2,2,6,6-tetramethylpiperidine 1-oxyl benzoate free radical, 2,2,6,6-tetramethyl-4-(2-propynyloxy)piperidine 1-oxyl free radical, 4-acetamido-2,2,6,6-tetramethylpiperidine 1-oxyl free radical, 4-cyano-2,2,6,6-tetramethylpiperidine 1-oxyl free radical, and 4-methoxy-2,2,6,6-tetramethylpiperidine 1-oxyl free radical.

A commercially available product may be used as the iodobenzene derivative. Examples thereof may include iodobenzene diacetate and [bis(trifluoroacetoxy)iodo]benzene.

### (Method of Controlling Inclusion Ratio of Cyclic Molecules of Aldehyde Group-Added Cyclic Molecule-Containing Polyrotaxane of the Present Disclosure)

The inclusion ratio of the cyclic molecules of the aldehyde group-added cyclic molecule-containing polyrotaxane may be controlled by appropriately selecting the kinds of its linear molecule and cyclic molecules.

The inclusion ratio is defined as the molar fraction of the aldehyde group-added cyclic molecules per number of repeating units in the linear molecule.

The number of the repeating units in the linear molecule may be calculated by, for example, dividing the molecular weight of the polymer by the molecular weight thereof per repeating unit.

The molar fraction of all the cyclic molecules in the linear molecule may be calculated by, for example, dividing the signal intensity (e.g., NMR spectrum) of the entirety of the cyclic molecules in the linear molecule (polyrotaxane) by a signal intensity (e.g., NMR spectrum) per one cyclic molecule.

A method for the calculation is described as described below with reference to, for example, Examples below.

The intensity of a signal derived from the cyclic molecules is 84H. When the intensity of a signal per one cyclic molecule is 7H, it can be calculated that 12 cyclic molecules are present (84H/7H=12).

The entirety of the linear molecule has 200 repeating units.

The inclusion ratio can be calculated to be (12/200)×100=6 mol % because the ratio is the ratio of the number of the cyclic molecules to the number of the repeating units of the entirety of the linear molecule.

Although the inclusion ratio of the present disclosure is not particularly limited, according to Examples below, the ratio is from 1 mol % to 40 mol %, preferably from 2 mol % to 15 mol %, more preferably from 2 mol % to 10 mol %.

Examples below have recognized that when the inclusion ratio is from 2 mol % to 10 mol %, the polyrotaxane is toughened (particularly in terms of fracture stress and toughness) as compared to an inclusion ratio in any other range.

(Biomaterial Subjected to Crosslinking Treatment with Polyrotaxane)

A biomaterial subjected to crosslinking treatment with a polyrotaxane of the present disclosure includes the following polyrotaxane:
(1) a linear molecule;
(2) capping groups (stopper molecules), provided that the capping groups are positioned at both the terminals of the linear molecule; and
(3) a cyclic molecule, provided that the inside of the cyclic molecule is penetrated by the linear molecule.

The cyclic molecule is preferably an aldehyde group-added cyclic molecule. Further, the aldehyde group-added cyclic molecule-containing polyrotaxane includes an aldehyde group-added cyclic molecule that is substantially free of a ketone group added thereto.

The phrase “substantially free of a ketone group added thereto” not only means that the cyclic molecule is completely free of a ketone group added thereto but also includes a case in which the number of ketone groups to be added is so small that there are no influences by the ketone groups. More specifically, according to the results of Examples below, the phrase means that 95% or more, 96% or more, 97% or more, 98% or more, or 99% or more of the hydroxy groups of the cyclic molecule are substituted with ketone groups.

Examples below have recognized that the biomaterial subjected to the crosslinking treatment with the polyrotaxane (in particular, thread-like collagen subjected to the crosslinking treatment with the polyrotaxane) of the present disclosure has stretchability.

### (Biomaterial)

The biomaterial of the present disclosure is not particularly limited as long as the biomaterial has a lysine residue. Examples thereof may include a protein (in particular, collagen), an enzyme, an antibody, and a peptide each having a lysine residue.

A preferred biomaterial may be, for example, thread-like collagen (in particular, thread-like atelocollagen). Although a method of producing the thread-like collagen is not particularly limited, the thread-like collagen may be produced by, for example, ejecting a collagen solution in a thread shape into a coagulating bath through an air gap to elongate and flow the collagen solution immediately before its spinning.

(Aspect of Biomaterial Subjected to Crosslinking Treatment with Polyrotaxane)

A preferred aspect of the biomaterial subjected to the crosslinking treatment with the polyrotaxane (in particular, a biomaterial subjected to crosslinking treatment with an aldehyde group-added cyclic molecule-containing polyrotaxane) of the present disclosure is as described below.

The linear molecule is a constituent unit based on polyethylene glycol, polypropylene glycol, or poly(ethylene glycol)-block-poly(propylene glycol)-block-poly(ethylene glycol).

The capping groups (stopper molecules) are each a constituent unit based on a triazine derivative or adamantanecarboxylic acid.

The aldehyde group-added cyclic molecule is a constituent unit based on an aldehyde group-added cyclodextrin.

The biomaterial is thread-like collagen.

The mechanical properties of the biomaterial, in which the linear molecule is a constituent unit based on polyethylene glycol, polypropylene glycol, or poly(ethylene glycol)-block-poly(propylene glycol)-block-poly(ethylene glycol), the capping groups (stopper molecules) are each a constituent unit based on a triazine derivative, and the aldehyde group-added cyclic molecule is a constituent unit based on an aldehyde group-added cyclodextrin, and which is thread-like collagen having stretchability, are as follows:
(1) a fracture stress of from 280 kPa to 1,300 kPa, from 280 kPa to 360 kPa, from 800 kPa to 1,300 kPa, or from 2,000 kPa to 3,200 kPa;
(2) a fracture strain of from 40% to 70%, from 62% to 70%, or from 40% to 52%; (3) an elastic modulus of from 18 kPa to 55 kPa, from 18 kPa to 34 kPa, from 39 kPa to 55 kPa, or from 100 kPa to 220 kPa;
(4) a toughness of from 83 kJ/m³ to 240 kJ/m³, from 83 kJ/m³ to 101 kJ/m³, from 100 kJ/m³ to 240 kJ/m³, or from 200 kJ/m³ to 350 kJ/m³; and
(5) a stress of from 10 kPa to 1,000 kPa at the time of the loading of a strain of from 30% to 40%.

The above-mentioned respective mechanical properties are measured by the following methods. A measurement sample is set in a commercial micro autograph (e.g., MST-X SYSTEM (Shimadzu Corporation)), and one of its ends is continuously pulled at a rate of 2 mm/min while its drying is prevented as required. The stress and strain of the sample are measured until the sample fractures.

In addition, the stress is calculated by dividing a test force detected in a tensile test by the sectional area of a thread (the sectional area of the thread is calculated from the equation “(radius)×(radius)×3.14” after the measurement of the diameter of the thread through observation with a microscope).

The above-mentioned biomaterial has stretchability because the biomaterial has one, two, three, four, or five of the above-mentioned properties (1) to (5).

(Method of Producing Biomaterial Subjected to Crosslinking Treatment with Polyrotaxane of the Present Disclosure)

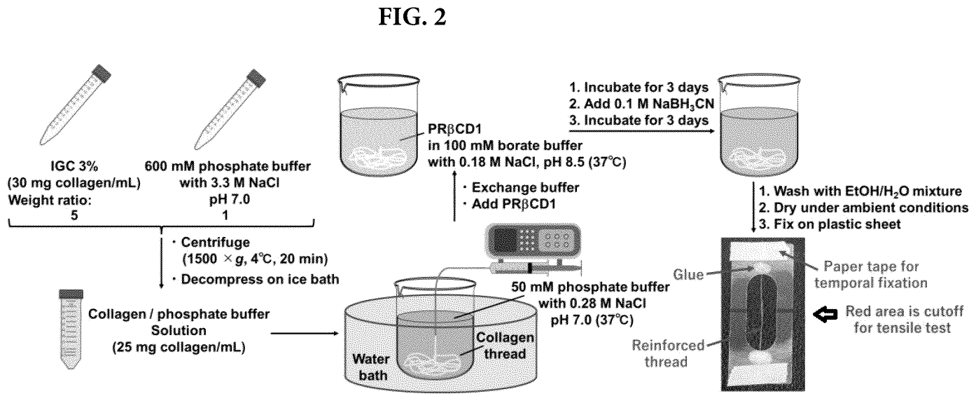

In a method of producing the biomaterial subjected to the crosslinking treatment with the polyrotaxane (in particular, the biomaterial subjected to the crosslinking treatment with the aldehyde group-added cyclic molecule-containing polyrotaxane) of the present disclosure, the following reductive amination reaction may be preferably performed under a mild condition (see FIG. 2).

Polyrotaxane-CHO+NH₂ of lysine residue-biomaterial=>polyrotaxane-CH₂—NH-Lys-biomaterial

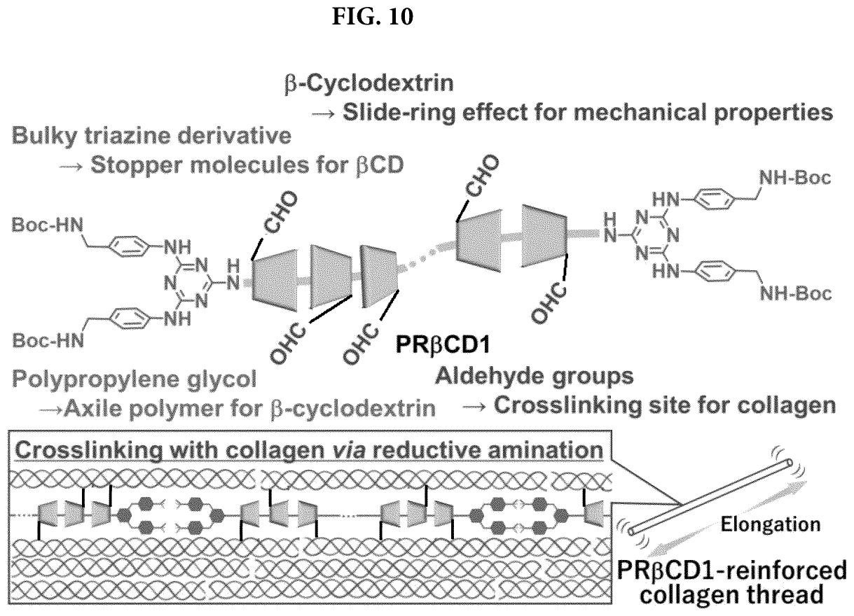

In the above-mentioned reaction, an aldehyde group of the polyrotaxane specifically and selectively reacts and crosslinks with the lysine residue of the biomaterial (see FIG. 10). Meanwhile, when crosslinking is performed by an amide coupling reaction through use of a general carboxyl group instead of an aldehyde group, nonspecific crosslinking (generally called an esterification reaction) occurs because the carboxyl group reacts with, for example, a serine or tyrosine residue. In addition, the properties of the biomaterial can be maintained because the reductive amination reaction can be performed under a mild condition.

The outline of the method of producing the biomaterial subjected to the crosslinking treatment with the polyrotaxane is described by taking thread-like collagen as an example.

A collagen solution is subjected to neutralization treatment and defoamed by extrusion into a buffer. Next, a collagen thread is immersed in a weakly basic buffer, and the polyrotaxane is added thereto, followed by shaking in the presence of a reducing agent. Finally, the surface of the thread is washed, and the thread is naturally dried, followed by fixation to a frame body. Thus, thread-like collagen subjected to crosslinking treatment with the polyrotaxane is produced.

The method of producing the biomaterial subjected to the crosslinking treatment with the polyrotaxane (in particular, the biomaterial subjected to the crosslinking treatment with the aldehyde group-added cyclic molecule-containing polyrotaxane) of the present disclosure preferably includes the following step:
(1) a step of subjecting a biomaterial having a lysine residue to reductive amination reaction treatment in the presence of a cyclic molecule (in particular, an aldehyde group-added cyclic molecule)-containing polyrotaxane.

Examples of the reducing agent to be used in the reductive amination reaction treatment may include sodium cyanoborohydride, sodium borohydride, lithium aluminum hydride, sodium triacetoxyborohydride, 2-picoline borane, and sodium dithionite that are known reducing agents.

Further, the biomaterial is preferably stored and subjected to the reaction in a buffer having a pH of from 5.0 to 10.0 (more preferably a pH of from 7.5 to 9.5) serving as a condition under which the stabilized state of the biomaterial (in particular, the thread-like collagen) and/or the polyrotaxane is kept. Examples of buffer may include acetic acid, phosphoric acid, carbonic acid, boric acid, and HEPES that are known buffers.

In addition, in the reductive amination reaction step, a hydride reducing agent (sodium cyanoborohydride (NaBH₃CN), sodium borohydride, aluminum lithium hydride, sodium triacetoxyborohydride, 2-picoline borane, or sodium dithionite) may be added to a reaction system for suppressing the production of a free aldehyde.

Conditions known per se may be adopted as a temperature, a pressure, a solvent, and the like in the above-mentioned step.

### (Crosslinking Composition)

A crosslinking composition of the present disclosure includes the aldehyde group-added cyclic molecule-containing polyrotaxane of the present disclosure as an effective component. In particular, the crosslinking composition of the present disclosure is not particularly limited as long as the composition is a material having an amino group. The composition is preferably used for, for example, a biomaterial, an organic material, or an inorganic material.

The present disclosure is described in more detail below by way of Experimental Examples. However, Experimental Examples below should be regarded as an aid for obtaining specific recognition of the present disclosure, and hence the scope of the present disclosure is by no means limited by Experimental Examples below.

### Example 1

### Material and Method

Materials and a method used in this Example are as described below.

### Experiment Method

### Materials

PPG-NH₂ (Mn: 4,000) serving as a constituent unit for a linear molecule was purchased from Sigma-Aldrich Co. LLC. Sodium chloride (NaCl), glutaraldehyde (GA), 2,4-dinitrophenylhydrazine (DNPH), and βCD serving as a constituent unit for a cyclic molecule were purchased from FUJIFILM Wako Pure Chemical Corporation. Anhydrous ethanol (EtOH, 99.5%), anhydrous tetrahydrofuran (THF), diethyl ether (Et₂O), 2,2,6,6-tetramethylpiperizine-1-oxyl (TEMPO), iodobenzene diacetate (PhI(OAc)₂), disodium hydrogen phosphate dodecahydrate (Na₂HPO₄·12H₂O), sodium dihydrogen phosphate dihydrate (NaH₂PO₄·2H₂O), and sodium cyanoborohydride (NaBH₃CN) were purchased from Kanto Chemical Co., Inc. Hexamethylphosphoric triamide (HMPA), PhI(OAcTf)₂, which was (bis(trifluoroacetoxy)iodo)benzene, Dess-Martin periodinane (DMP), and N,N-diisopropylethylamine (DIPEA) were purchased from Tokyo Chemical Industry Co., Ltd. All the reagents were special grades, and were used as they were without being further purified after their purchase. The compound of the formula (1) serving as a constituent unit for a stopper molecule was synthesized in accordance with a method described in the literature “Org. Lett. 2000, 2 (6), 843-845.”

A 3% aqueous solution (30 mg/mL) of medical type I atelocollagen produced and sold by the applicant was used in the production of thread-like atelocollagen of the present disclosure.

### (Synthesis 1 of Aldehyde Group-Added Cyclic Molecule-Containing Polyrotaxane of the Present Disclosure)

The polyrotaxane of the present disclosure was synthesized in accordance with a synthesis scheme illustrated in FIG. 1. Details thereof are as described below.

### Synthesis of PRβCD3

PPG-NH₂ (0.50 g, 0.125 mmol) was loaded into a 1,000-milliliter beaker. Subsequently, an aqueous solution containing water (600 mL) and βCD (6.6 g, 5.8 mmol) was added to the beaker, and the mixture was stirred at 25° C. for 7 days. Thus, a white precipitate was obtained. The precipitate was centrifuged and then freeze-dried to provide a PRβCD3 as a white solid in 88% yield (3.2 g, 0.11 mmol).

δH (DMSO-d₆): 0.85-1.04 (3H, —CH₃, PPG), 3.55-3.57 (14H, βCD), 3.61-3.66 (28H, βCD), 4.46 (7H, βCD), 4.82 (7H, βCD), 5.70-5.76 (14H, βCD).

### Synthesis of PRβCD2

The stopper molecule (2.0 g, 3.6 mmol) represented by the chemical formula (1) was dissolved in a solution of DIPEA (2.0 mL, 11.5 mmol) and dry THF (5.0 mL). PRβCD3 powder (1.0 g, 0.034 mmol) was gradually added to the solution containing the stopper molecule, and the suspension was vigorously stirred at 25° C. for 4 days. After that, the suspension was sufficiently washed with cold THF, and was centrifuged until the supernatant became colorless. The resultant solid was vacuum-dried, and was dialyzed with a dialysis membrane (Spectra/Por™ 7, MWCO=1 kDa) in water for 3 days. The dialysis fluid in the membrane was freeze-dried to provide a PRβCD2 as a white solid (0.47 g, 47 wt %).

Calculation from a ratio between the respective characteristic signals recognized that the number of βCD molecules for forming one PRβCD2 molecule was from 23 to 25.

δH (DMSO-d₆): 0.85-1.04 (3H, —CH₃, PPG), 1.47 (9H, —C(CH₃)₃, Boc), 3.54-3.57 (14H, βCD), 3.61-3.65 (28H, βCD), 4.47 (7H, βCD), 4.83 (7H, βCD), 5.69-5.74 (14H, βCD), 7.32-7.48 (4H, benzene ring, stopper moiety), 7.64 (1H, —NH-Boc, stopper moiety), 9.30 (1H, Ar—NH-Trz, stopper moiety), 10.1 (1H, Ar—NH-Trz, stopper moiety). IR (KBr): v=3,383, 1,155, 1,080, 1,032 cm⁻¹.

### Reaction Between βCD and DMP

An attempt was made to synthesize βCD monoaldehyde in accordance with a method in the previous report (reference: Tetrahedron Lett. 1995, 36 (46), 8371-8374.). Specifically, βCD (1.0 g, 0.88 mmol) and 2 equivalents of DMP (0.75 g, 1.76 mmol) that had just been unsealed were dissolved in DMSO (25 mL), and the solution was stirred at 25° C. for 1 hour. After that, the reaction mixture was poured into cold acetone (150 mL), and the mixture was cooled to −10° C. to precipitate a crude product. The precipitation step was repeated until a DMP by-product was completely removed. Subsequently, the resultant was extracted with water and freeze-dried to provide oxidized βCD as white powder (0.9 g, 90%). Both the primary and secondary alcohol moieties of βCD were oxidized. Accordingly, the product became a mixture, and was hence not able to be isolated.

A peak of a ¹H NMR spectrum was assigned with respect to pure βCD.

δH (D₂O): 3.45-3.57 (m, 14H), 3.73-3.89 (m, 28H), 4.96-5.04 (m, 7H), 5.31-5.43 (m, acetal (—CH(OD)₂, —CH(OH)(OD), —CH(OH)₂)). δ¹³C (D₂O): 60.3, 71.8, 72.1, 73.1, 81.1, 101.9.

### Reaction Between βCD and TEMPO/PhI(OAc)₂ Redox Couple

βCD (1.0 g, 0.88 mmol), a catalytic amount of TEMPO (0.014 g, 0.09 mmol), and PhI(OAc)₂ (0.28 g, 0.88 mmol) were dissolved in dry DMF (25 mL), and the solution was stirred at 25° C. for 24 hours. After that, the crude mixture was added to Et₂O (500 mL), and the whole was cooled at 0° C. overnight. Subsequently, the precipitate was filtered, and was sufficiently washed with acetonitrile. The washed product was extracted with water, and was subsequently freeze-dried to provide βCD monoaldehyde as a white solid (0.85 g, 85%).

δH (D₂O): 3.47-3.58 (m, 14H), 3.76-3.90 (m, 28H), 4.98 (d, 7H), 5.30-5.36 (m, 1H, acetal (—CH(OD)₂, —CH(OH)(OD), —CH(OH)₂)). δ¹³C (D₂O): 60.3, 71.8, 72.1, 73.1, 101.9.

### Synthesis of PRβCD1

The PRβCD2 (100 mg, 3.3×10−3 mmol) containing about 23 βCD molecules (7.6×10⁻² mmol) was dissolved in cold HMPA (5 mL) containing DIPEA (200 μL, 1.2 mmol) at 4° C. Subsequently, a catalytic amount of TEMPO (2.4 mg, 1.5×10⁻² mmol) and PhI(OAcTf)₂ (33 mg, 7.6×10⁻² mmol) were added to the solution, and the mixture was vigorously stirred at 4° C. for 10 days. Next, the solution was dropped into an excessive amount of cold Et₂O to precipitate a crude product, and the crude product was repeatedly washed by being centrifuged (1,500×g) in cold acetonitrile. Finally, the resultant solid was dialyzed with a Spectra/Por™ 7 dialysis membrane (MWCO: 1 kDa) in water for 2 days. The resultant solution was freeze-dried to provide a PR CD1 as a white solid (40 mg, 40 wt %).

δH (D₂O): 1.20 (3H, —CH₃, PPG, and Boc), 2.45-2.59 (3H, PPG), 3.08 (1H, α-hydrogen, acetal), 3.49-3.59 (14H, βCD), 3.72-4.00 (28H, βCD), 4.94 (7H, βCD), 5.23-5.38 (1H, acetal (—CH(OD)₂, —CH(OH)(OD), —CH(OH)₂)). δ¹³C (D₂O): 12.2, 16.3, 17.8, 34.5, 42.6, 54.4, 60.4, 62.6, 71.6, 71.8, 71.8, 72.0, 72.1, 73.1, 76.5, 77.2, 78.2, 78.3, 80.3, 80.7, 80.8, 80.9, 81.1, 81.3, 98.0, 98.1, 100.8, 101.3, 101.4, 101.5, 101.7, 101.8, 101.9. IR (KBr): v=3,383, 1,209, 1,155, 1,080, 1,032 cm⁻¹.

### (Production of Thread-Like Atelocollagen of the Present Disclosure)

The thread-like atelocollagen (Col-PRβCD1 thread) of the present disclosure was produced in accordance with a synthesis scheme illustrated in FIG. 2. Details thereof are as described below.

Specifically, 15.0 g of the 3% atelocollagen solution and 3.0 g of a 600 mM phosphate buffer (containing 3.3 M NaCl, pH: 7) were mixed. The mixture was stirred in an ice bath, and was then deaerated with a centrifugal separator (1,500×g, 4° C., 20 minutes). After that, decompression treatment was performed in the ice bath. The deaeration was continued until all the air bubbles disappeared. The resultant viscous solution was taken in with an 18-gauge disposable syringe, and was extruded into a warmed (37° C.) 50 mM phosphate buffer (200 mL, pH: 7) containing 0.28 M NaCl through an 18-gauge elastic tube (25 cm) over 15 minutes. To crosslink atelocollagen molecules, a stepwise reductive amination method (i.e., imination and subsequent imine reduction) was adopted. First, a thread to be crosslinked was immersed in a buffer (warmed to 37° C.) of the PRβCD1 having aldehyde groups whose number of moles was equal to the total number of the lysine residues of the atelocollagen (i.e., 102 equivalents in terms of lysine residue). A crosslinking reaction was continued in a 100 mM borate buffer of the thread containing 0.18 M NaCl (pH: 8.5), and the imination was quantitatively recognized from the colorimetric detection of an aldehyde with DNPH. After the imination, NaBH₃CN was added so that its final concentration became 0.1 M, followed by the holding of the resultant solution at 37° C. for 3 days. Thus, reductive amination was advanced so that the crosslinking was stabilized.

As a comparative control in the evaluations of mechanical properties, a general-purpose aldehyde-based crosslinking agent glutaraldehyde (GA) was also used in the crosslinking of the atelocollagen.

### (Tensile Test)

A thread-like atelocollagen sample was immersed in a 50 mM phosphate buffer (pH: 7) containing 0.28 M NaCl for 2 minutes. The top and bottom of the thread were pinched with the jigs of a micro autograph, and as illustrated in FIG. 2, the left and right red areas of a plastic sheet were removed to enable the measurement of the strength of the fixed thread. In a tensile test, while the sample was exposed to water mist for preventing the drying of the sample, the upper portion of the thread was continuously pulled at a rate of 2 mm/min, and the stress and strain of the sample were measured until the sample fractured. The statistical significance of experimental data was evaluated by Tukey's test, and a p-value of less than 0.01 was regarded as significant.

### (Apparatus)

A high-speed refrigerated centrifuge (CR21GIII, Hitachi, Ltd.) was used in the purification of a prepared compound. An FDU-2200 freeze dryer (EYELA) including a vacuum pump was used in the freeze drying of a synthesized compound. A thermostat THERMO MINDER 50 (TAITEC) and an SDPC-1 syringe pump (AS ONE Corporation) were used in the production of the thread-like atelocollagen. The crosslinking of the thread-like atelocollagen was performed with an FF-12 incubator (Fine) at 37° C. A micro autograph MST-X SYSTEM (Shimadzu Corporation) was used in the measurement of the mechanical properties. NMR measurement was performed with an ECS-400, ECA-500, or ECZ-600R NMR spectrometer (JEOL Ltd.).

### Example 2

### (Synthesis Result of Aldehyde Group-Added Cyclic Molecule-Containing Polyrotaxane)

### Synthesis of PRβCD2

The stopper molecules were synthesized in the same yield as that achieved in the previous report (i.e., about 95%). Chlorine atoms bonded to carbon, the atoms remaining in the stopper molecules, substituted the terminal amino groups of a pseudo-PR. The modification of the terminals with the stopper molecules under a normal temperature condition was able to provide the PRβCD2 in about 50% yield. Some previous studies have reported that the heating of a reaction mixture (at 60° C. or 80° C.) achieves the same reaction, and hence enables efficient synthesis of a triazine derivative (https://doi.org/10.1016/B978-008096519-2.00042-4). However, when the pseudo-PR (PRβCD3) was subjected to a reaction at high temperature, the synthesis yield of the triazine derivative significantly reduced (<1 wt %).

### (Synthesis of βCD Monoaldehyde)

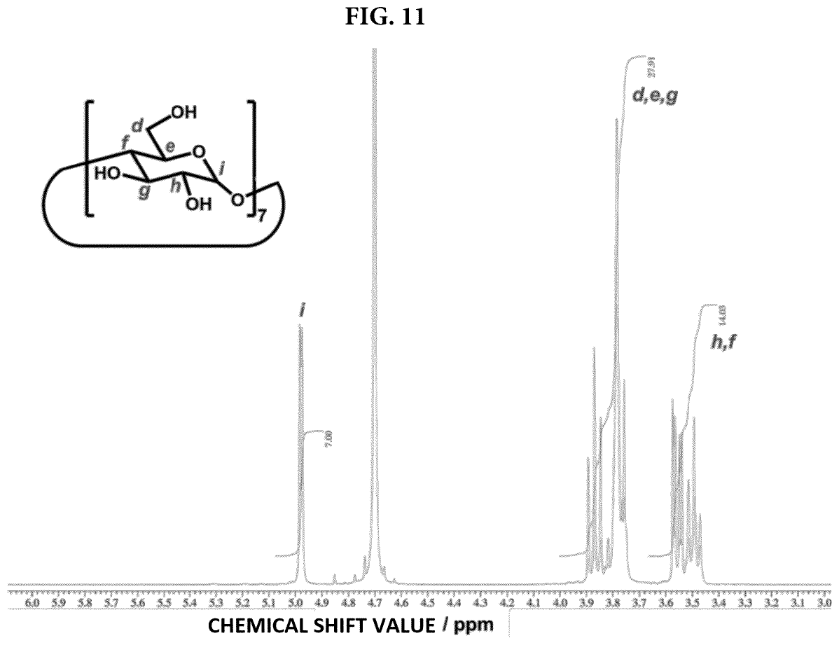

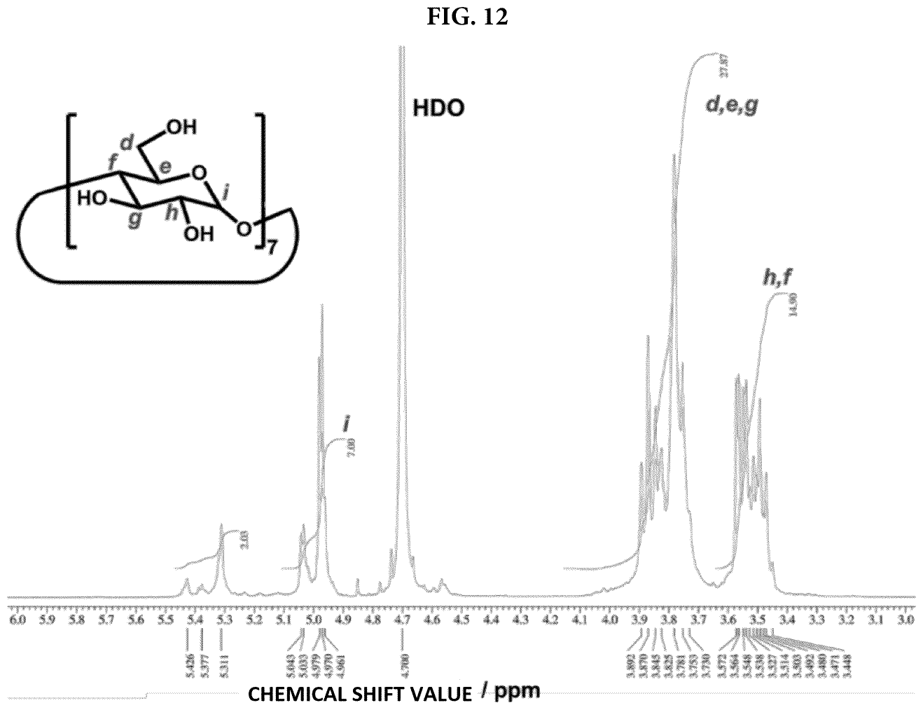

Before the introduction of one aldehyde group per one βCD molecule of a PR, an oxidation reaction test was performed by using only DMP and βCD. To directly detect an aldehyde group, first, ¹H NMR measurement was performed in DMSO-d₆. However, the intensity of an aldehyde group-derived signal changed during several times of measurement. It was conceived from the foregoing that the reactivity of an aldehyde group (e.g., the formation of an acetal with a residual hydroxy group of βCD) inhibited quantitative analysis. In view of the foregoing, the use of the ¹H NMR spectrum of oxidized βCD measured in D₂O in comparison revealed that a large change occurred in the chemical structure of βCD (FIG. 11 and FIG. 12). Specifically, a signal that was able to be assigned to Ci-H (Hi) partially shifted to lower magnetic fields as compared to a signal in the case of βCD alone. In addition, a new signal was detected at from 5.31 ppm to 5.43 ppm, and was considered to be a spectral change due to the presence of a ketone or an aldehyde in the structure of βCD. It has been known that DMP oxidizes primary and secondary alcohols. Accordingly, DMP can oxidize the Cd-OH, Cg-OH, and Ch-OH groups of βCD. From the viewpoint of general organic chemistry, the production of a ketone in Cg or Ch partially shifts the signal of the Hi close thereto to lower magnetic fields. The signal at from about 5.31 ppm to about 5.43 ppm formed of three peaks can be assigned to three kinds of acetals produced from an aldehyde (i.e., —CH(OD)₂, —CH(OH)(OD), and —CH(OH)₂). The chemical shift of a peak related to an acetal falls within a range that has already been reported for a synthetic acetal.

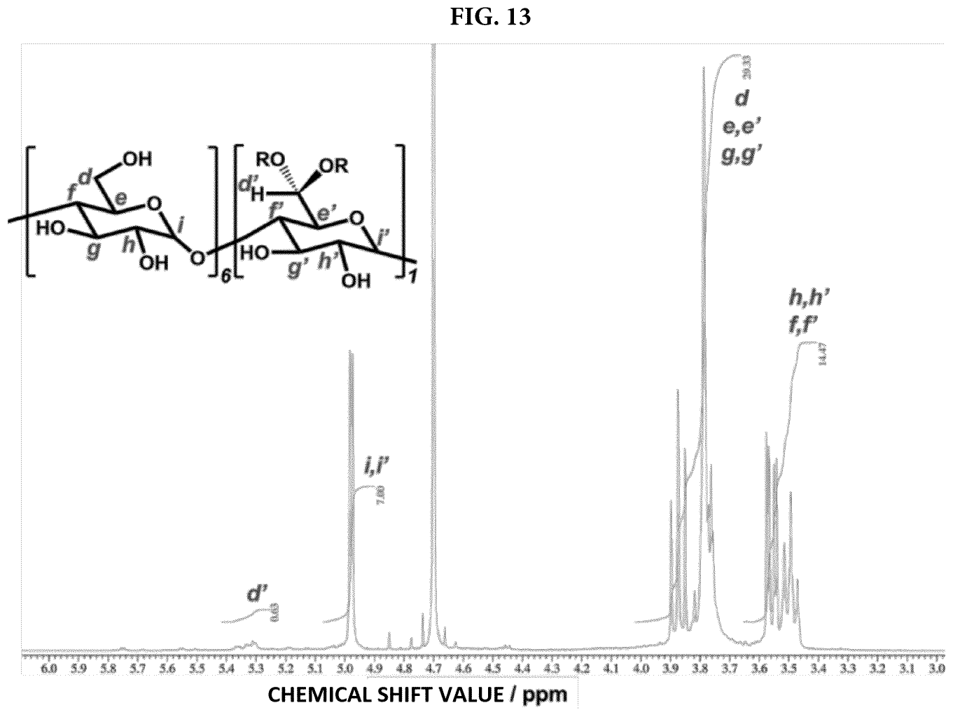

Meanwhile, in oxidation with a TEMPO/PhI(OAc)₂ redox couple, no change in shape of an Hi signal was observed (FIG. 11 and FIG. 13). In addition, the spectral shapes and intensities of the other proton species substantially coincided with those of βCD serving as a raw material that had not been subjected to any oxidation treatment (FIG. 11 and FIG. 13). Those results suggested that Cg-OH and Ch-OH remained substantially unreacted. That is, it can be said that the primary alcohol moiety of βCD was selectively subjected to the reaction. Depending on reaction conditions, a primary alcohol and a secondary alcohol can be oxidized with TEMPO. However, owing to steric hindrance caused by the two dimethyl groups of TEMPO, the primary alcohol (i.e., the Cd-OH of βCD) was preferentially oxidized to form an aldehyde.

Accordingly, the TEMPO/PhI(OAc)₂ redox couple is more suitable for the selectively production of an aldehyde in βCD than DMP is.

### (Synthesis of PRβCD1)

On the basis of the above-mentioned results, first, an aldehyde group was selectively introduced into the PRβCD1 with the TEMPO/PhI(OAc)₂ redox couple. When the reaction was performed in a typical polar solvent (DMF or DMSO), the yield of the PRβCD1 was low (<0.1 wt %). Such low yield can be described from the viewpoint of a reaction mechanism. More specifically, as the oxidation of a hydroxymethyl group advances, a reaction solution becomes more acidic. The acidification protonates the nitrogen atoms of the stopper molecules to enhance the electrophilicity of a triazine ring, to thereby accelerate a nucleophilic substitution reaction with a potential nucleophile (e.g., DMF, DMSO, or an acetate anion) in the reaction solution. As a result of the nucleophilic substitution reaction, the bulkiness of each of the stopper molecules was lost, and hence βCD was released from the PRβCD2 to significantly reduce the synthesis yield.

To avoid the problem, PhI(OAcTf)₂ was used as an oxidizing agent, and an oxidation reaction was performed in HMPA in the presence of abase (DIPEA). Stirring at 4° C. for 10 days increased the yield of the PRβCD1 to 40 wt %.

### (NMR Analysis of PRβCD1)

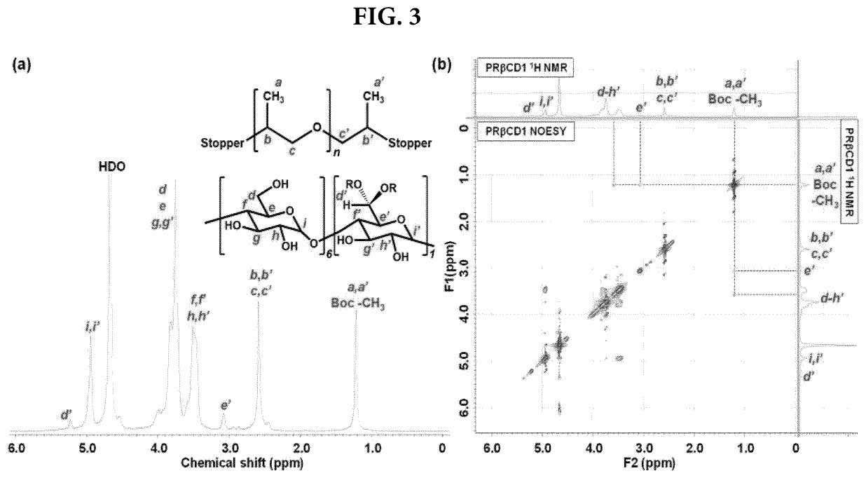

The ¹H NMR spectrum of the PRβCD1 is shown in FIG. 3a. In the figure, it can be recognized that the intensity of a signal at from 2.45 ppm to 2.59 ppm is substantially equal to the intensity of a signal assigned to each of the methyl groups of PPG and Boc at 1.20 ppm. The signal was able to be assigned to each of the Hb, Hb′, Hc, and Hc′ atoms of the PPG. Those signals were detected in a relatively upfield region as compared to that in the case of free PPG. This may result from the fact that amphiphilic PPG is incorporated into a βCD structure. In addition, signals at from 3.49 ppm to 3.59 ppm and from 3.72 ppm to 4.00 ppm were assigned to βCD (Hd-h′). It was calculated from a ratio between the signals related to Ha,a′ and the Hd-h′ that the PRβCD1 included about 25 βCD molecules. An intensity ratio between the Ha,a′ and Hi,i′ provided the same number of βCD molecules per PRβCD1. Interestingly, a new signal was detected at 3.08 ppm. It was suggested from an intensity ratio between the signal and the Ha,a′ signal that a chemical species having an intensity corresponding to one H atom was present. The signal at 3.08 ppm was considered to be derived from an α-hydrogen atom (He′) bonded to an acetal because Hd′ was able to be assigned to each of acetal species detected in βCD monoaldehyde (i.e., —CH(OD)₂, —CH(OH)(OD), and —CH(OH)₂). The acetal species are each formed by the hydration of an aldehyde group, and hence the number of aldehyde groups in the PRβCD1 may be calculated by using the intensity of the He′ that is an acetal-derived peak. As a result of the calculation of the number of the aldehyde groups, the number was estimated to be about 25. The number coincides well with the number of the βCD molecules in the PRβCD1. That is, it can be said that one aldehyde group is added per one βCD molecule. Further, in consideration of the detection sensitivity of NMR, it was revealed that the above-mentioned aldehyde group addition reaction (synthesis of the PRβCD1) advanced with an efficiency of 95% or more.

Further, a negative NOE correlation was observed in each of the combinations of the Ha,a′ and the He′, and of the Ha,a′ and the Hd-h′ (FIG. 3b).

As can be seen from the foregoing, it was recognized from the results of one-dimensional and two-dimensional NMR measurements that a PPG unit was included in the βCD structure. Further, water solubility as a result of the success of the formation of a PR structure in an aqueous solution was recognized.

### Example 3

### (Identification of Mechanical Properties of Thread-Like Atelocollagen of the Present Disclosure)

After the recognition of the success of the synthesis of the PRβCD1, the thread-like atelocollagen {PRβCD1-reinforced atelocollagen thread (Col-PRβCD1)} of the present disclosure was produced by a reductive amination method. The influences of a PR structure on the mechanical properties of a thread to be obtained as a result were examined by using GA serving as a conventional aldehyde-based crosslinking agent.

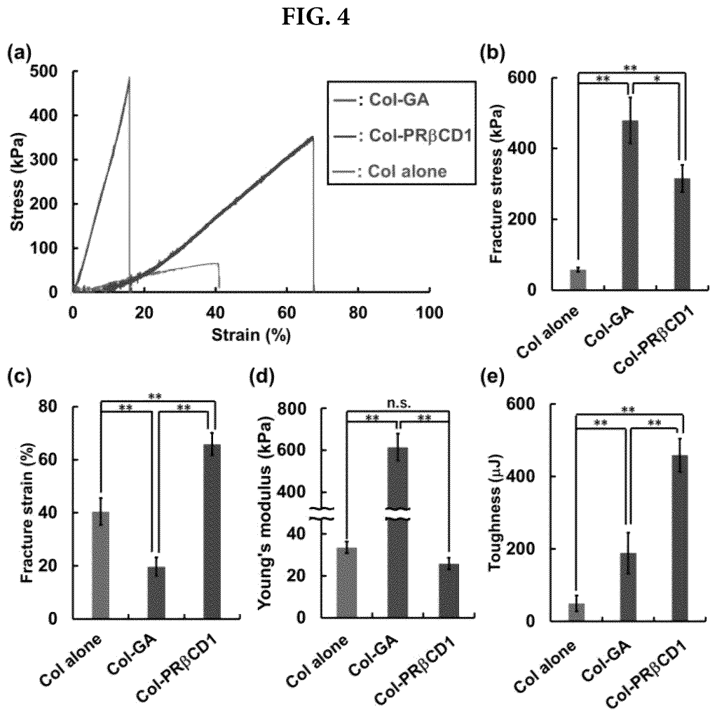

As shown in FIG. 4b, when the thread was crosslinked with GA, its fracture stress significantly increased from 60(±6) kPa to 480(±65) kPa. Meanwhile, the fracture strain of the thread reduced from 40%(±5%) to 20%(±3.5%) as compared to that in the case of Col alone (FIG. 4c). In addition, the Young's modulus thereof was determined from the initial region of a stress-strain curve in which the stress and the strain linearly correlated with each other. As shown in FIG. 4d, the Young's modulus increased from 34(13) kPa to 570(145) kPa after the crosslinking, that is, the Young's modulus became significantly high. Increases in fracture stress and Young's modulus, and a reduction in fracture strain are each typical behavior of chemical crosslinking. The area of the stress-strain curve representing the toughness of Col-GA (i.e., 190(±60) μJ) increased to 3.8 times as large as that in the case of the Col alone (i.e., 50(±22) μJ) (FIG. 4e).

The stress-strain curve of the thread-like atelocollagen (Col-PRβCD1) was significantly different from that of the Col-GA or the Col alone. Specifically, the Col-PRβCD1 thread showed a J-shaped stress-strain curve typically observed in a PR-based crosslinked material (FIG. 4a). When the thread was crosslinked with the PRβCD1, both of its fracture stress and fracture strain increased. The fracture stress increased by a factor of 5.4 (320(±40) kPa), and the fracture strain increased by a factor of 1.6 (66%(±4%)) (see FIG. 4a to FIG. 4c). As a result, the toughness of the Col-PRβCD1 was 92(±9) kJ/m³, that is, the Col-PRβCD1 showed the highest value among the tested threads (FIG. 4e). The Young's modulus of the thread was substantially insusceptible to the crosslinking with the PRβCD1 (26(18) kPa) (FIG. 4d).

As a whole, the mechanical properties observed for the Col-PRβCD1 showed the slide-ring features of a PR.

### Example 4

### (Identification of Stress of Thread-Like Atelocollagen of the Present Disclosure)

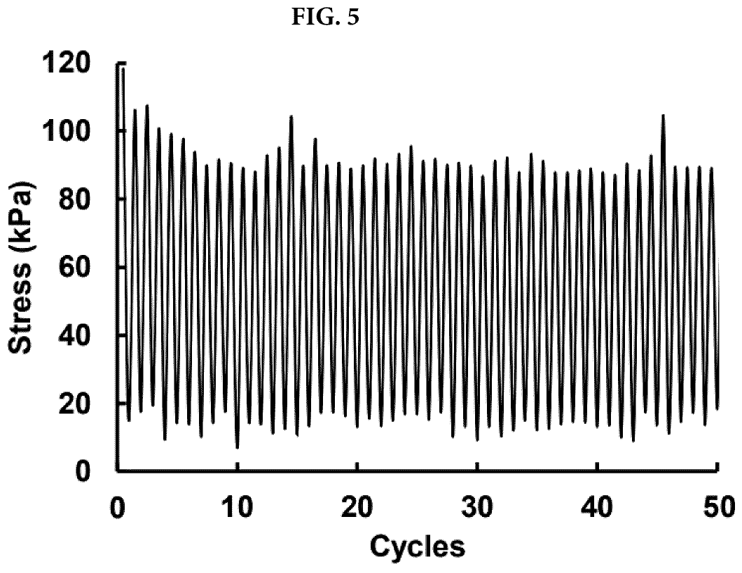

The stress of a thread at the time of repeated application of a strain load to the thread-like atelocollagen of the present disclosure was measured (FIG. 5). The strain thereof was set to from 30% to 40% in terms of relative value.

As a result, it was found that the stress of the thread changed in the range of from 10 kPa to 100 kPa in a roughly constant manner. The result shows that the thread-like atelocollagen of the present disclosure maintains its mechanical property in a relative strain range in which the Col alone fractures (FIG. 4c).

Accordingly, the thread-like atelocollagen of the present disclosure has a stretchable property, and hence can be adopted as a biomaterial that can be used for a long time period in, for example, the field of regenerative medicine.

### Example 5

### (Synthesis 2 of Aldehyde Group-Added Cyclic Molecule-Containing Polyrotaxane of the Present Disclosure)

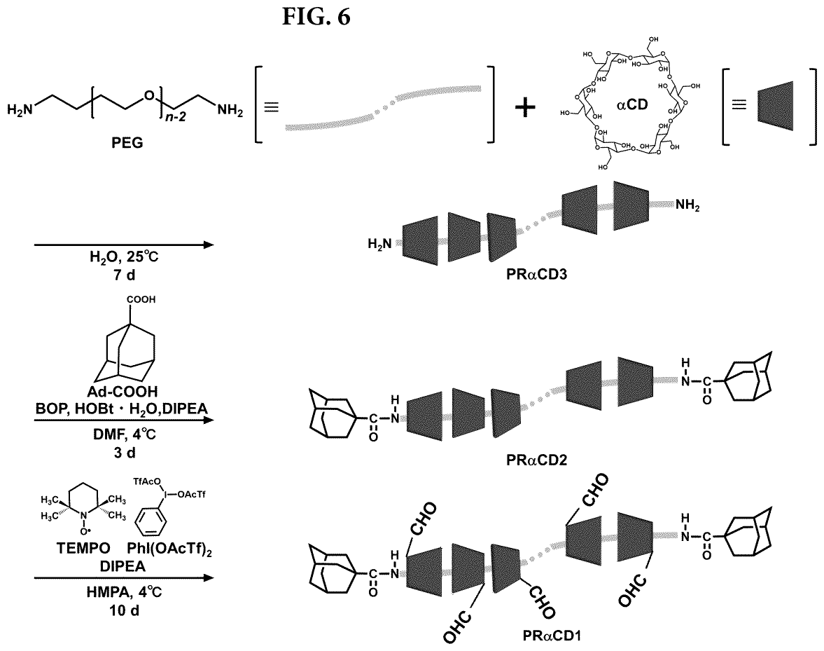

In this Example, an aldehyde group-added cyclic molecule-containing polyrotaxane was synthesized on the basis of the description in Example 1 except that: PEG₅₀₀ₖ-NH₂ was used as a constituent unit for a linear molecule instead of PPG-NH₂ (Mn: 4,000); and α-cyclodextrin was used instead of β-cyclodextrin (FIG. 6). PEG₅₀₀ₖ-NH₂ was produced by using a production method known per se (reference: Chem, 2016, 1, 766).

### (Synthesis of PRαCD3)

PEG₅₀₀ₖ-NH₂ (0.5 g, 0.001 mmol) and α-cyclodextrin (αCD) (5.0 g, 5.1 mmol) were stirred in 100 mL of pure water at 25° C. for 7 days. After the stirring, to collect the resultant white precipitate, the mixture was centrifuged (1,500×g, 25° C., 10 minutes). The supernatant was removed, and the white precipitate was freeze-dried to provide 3.0 g of a PEGαCD3. The synthesis of the PRαCD3 was recognized by ¹H NMR.

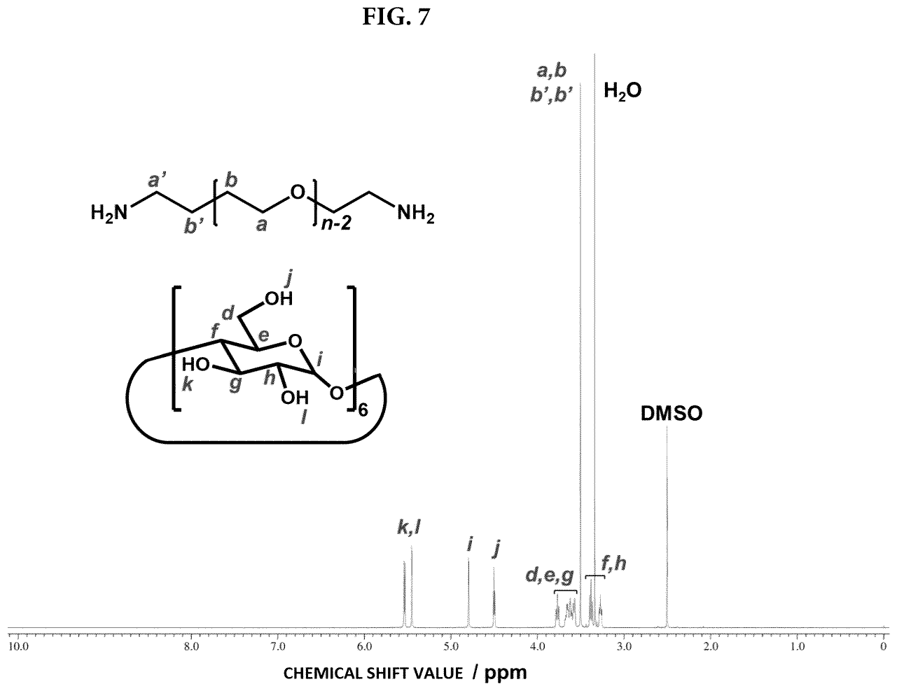

The ¹H NMR spectrum of the PRαCD3 is shown in FIG. 7. The synthesis of the PRαCD3 was able to be recognized because the spectra of the PEG and αCD were observed. Meanwhile, in an organic solvent such as DMSO, the PRαCD3 was observed under such a state that its polyrotaxane structure dissociated because the PRαCD3 was free of stopper molecules at both of its terminals.

### (Synthesis of PRαCD2)

The PEGαCD3 (1.0 g, 4.3×10⁻⁴ mmol), DIPEA (0.15 mL, 0.86 mmol), BOP (0.38 g, 0.27 mmol), which was 1H-benzotriazol-1-yloxytris(dimethylamino)phosphonium hexafluorophosphate, Ad-COOH (0.16 g, 0.89 mmol), which was adamantane carboxylic acid, and HOBt-H₂O (0.12 g, 0.89 mmol), which was 1-hydroxybenzotriazole monohydrate, were dissolved in 2.3 mL of DMF cooled to 4° C., and the solution was stirred for 3 days. After the reaction, the reaction liquid was dropped into diethyl ether, and the resultant precipitate was repeatedly subjected to washing with acetonitrile and centrifugation (1,500×g, 4° C., 10 minutes) so that DIPEA, BOP, and Ad-COOH that had been excessively added were removed. Thus, a PRαCD2 was obtained. The synthesis of the PRαCD2 was recognized by ¹H NMR.

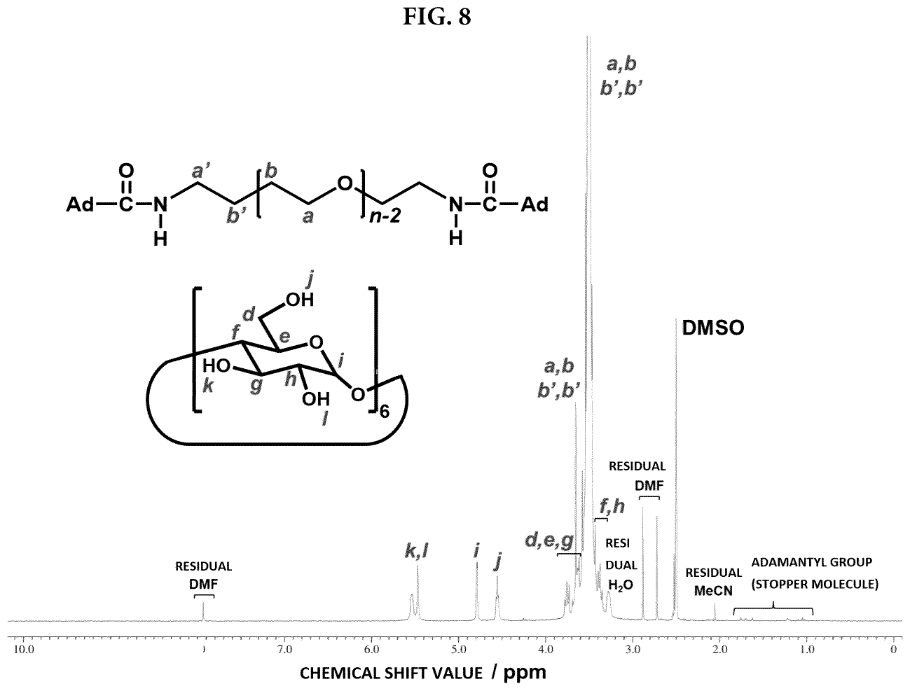

The ¹H NMR spectrum of the PRαCD2 is shown in FIG. 8. The peaks of polypropylene glycol and αCD were observed, and the peaks broadened. Further, the peak of an adamantyl group was observed. It was able to be recognized from those results that the PRαCD2 was synthesized. Although a plurality of kinds of residual solvents were detected, the solvents were used as they were because the solvents did not affect the synthesis of a PRαCD1 in principle.

### (Synthesis of PRαCD1)

The PEGαCD2 (0.05 g, 2.2×10⁻⁵ mmol), TEMPO (2.0 mg, 1.3×10⁻² mmol), PhI(OAcTf)₂ (18 mg, 4.2×10⁻² mmol), and DIPEA (0.1 mL, 0.57 mmol) were dissolved in 1.7 mL of HMPA cooled to 4° C., and the solution was stirred for 10 days. After the reaction, the reaction liquid was dropped into diethyl ether, and the resultant precipitate was repeatedly subjected to washing with acetonitrile and centrifugation (1,500×g, 4° C., 10 minutes), followed by drying under reduced pressure. Thus, 0.03 g of the PRαCD1 was obtained. The synthesis of the PRαCD1 was recognized by ¹H NMR.

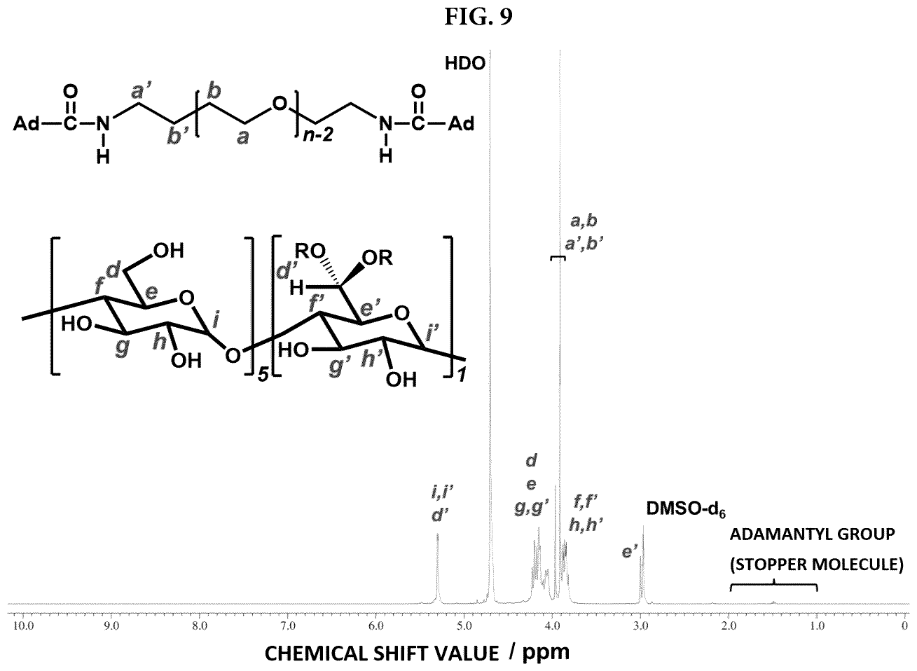

The ¹H NMR spectrum of the PRαCD1 is shown in FIG. 9. Peaks derived from the PEG and αCD were observed, and peaks (d′ and e′) derived from acetals produced by the hydration of aldehydes were also observed. It was recognized from those results that the PRαCD1 was synthesized. In addition, the introduction ratio of αCD per one PRαCD1 molecule was calculated to be 25 mol % from the integration ratio of the spectrum. In other words, it was recognized that about 3,000 αCD molecules were incorporated per PRαCD1 single strand.

### Example 6

A Col-PRαCD1 serving as thread-like atelocollagen was produced from the PRαCD1 produced in Example 5 by the production method described in the above-mentioned section “Production of Thread-like Atelocollagen of the Present Disclosure.” Further, the mechanical properties of the Col-PRαCD1 were identified by the method described in the above-mentioned section “Tensile Test.”

The results of the identification are shown in FIG. 4 and Table 1 below.

As compared to the Col-PRβCD1, the fracture stress of the Col-PRαCD1 increased to 1,050±50 kPa. The elastic modulus thereof increased to 47±8 kPa. The toughness thereof increased to 170±70 kJ/m³. Meanwhile, the fracture strain thereof reduced to 46±6%, that is, the fracture strain was comparable to that of the Col alone. It was recognized from those measurement results that the Col-PRαCD1 had stretchability as in the Col-PRβCD1.

TABLE 1

| | Col alone | Col-GA | Col-PRβCD1 | Col-PRαCD1 |
|---|---|---|---|---|
| Fracture | 60 ± 6 | 480 ± 65 | 320 ± 40 | 1,050 ± 250 |
| stress | | | | |
| (kPa) | | | | |
| Fracture | 40 ± 5 | 20 ± 3.5 | 66 ± 4 | 46 ± 6 |
| strain | | | | |
| (%) | | | | |
| Elastic | 34 ± 3 | 570 ± 45 | 26 ± 8 | 47 ± 8 |
| modulus | | | | |
| (kPa) | | | | |
| Toughness | 10 ± 4 | 38 ± 11 | 92 ± 9 | 170 ± 70 |
| (kJ/m³) | | | | |

### Example 7

In this Example, inclusion ratio-controlled polyrotaxanes (a Plu₁₅βCD1 and a Plu₉ₖβCD1) of the present invention were synthesized. Details thereof are as described below.

Hydroxyl group-terminal Pluronic™ reagents (Plu₉ₖ-OH and Plu₁₅ₖ-OH) were purchased from Sigma-Aldrich Co. LLC. Carboxyl group-terminal Pluronic™ compounds (Plu₉ₖ-COOH and Plu₁₅ₖ-COOH) were synthesized through bleach oxidation that was a known method (Chem. Lett. 2016, 45, 991-993.). Tritylamine was purchased from Tokyo Chemical Industry Co., Ltd. A Spectra/Por™ dialysis membrane (MWCO: 1 kDa) was used in purification in each step.

### (Formation of Pseudopolyrotaxane Through Use of Both-Terminal Carboxylic Acid Pluronic™ (Synthesis of PluPRβCD3))

Pluronic-COOH (1 g) and β-CD (1 g, 0.88 mmol) were stirred in 56 mL of pure water for 1 week. A white precipitate produced by the stirring was centrifuged (14,000×g, 20° C., 30 minutes), and was washed with cold water, followed by centrifugation again. The resultant was finally freeze-dried to provide a white solid.

δH (DMSO-d₆): 1.01-1.05 (m, 3H, Cⱼ—H), 3.30-3.38 (m, C_{b,d}—H and C_{k,l}—H), 3.41-3.67 (m, 32H, C_{c,e,f}—H and Cₘ—H), 3.94 (br, 4H, Cₙ—H), 4.37-4.40 (m, 7H, OHg), 4.83-4.84 (m, 7H, Cₐ—H), 5.63-5.68 (m, 14H, OH_{h,i})

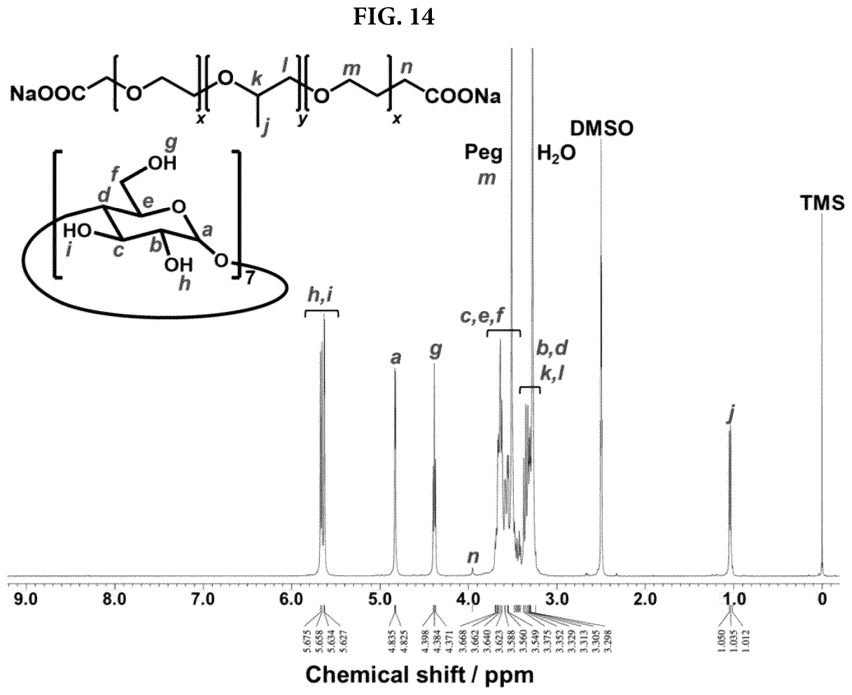

The formation of a PluPRβCD3 was recognized because peaks derived from Pluronic and β-cyclodextrin were observed in the ¹H NMR spectrum of FIG. 14. In addition, the inclusion ratio of the pseudopolyrotaxane was calculated to be 6 mol % from an intensity ratio between the Pluronic-derived signal (j) and the β-cyclodextrin-derived signal (a).

A method of calculating the inclusion ratio is as described below.

The intensity of the signal (j) was 90H per one Pluronic chain, and hence the portion was normalized to 90H. At this time, the intensity of the β-cyclodextrin-derived signal (a) became 84H. It was calculated that 12 β-cyclodextrin molecules were present (84H/7H=12) because the intensity of the signal (a) per one β-cyclodextrin molecule was 7H. Meanwhile, the entirety of the Pluronic chain has 200 repeating units (85×2+30=200). The inclusion ratio was calculated to be (12/200)×100=6 mol % because the ratio was the ratio of the number of the β-cyclodextrin molecules to the number of the repeating units of the entirety of the Pluronic chain.

### (Introduction of Tritylamide Capping Group (Synthesis of PluPRβCD2))

Tritylamine (0.2 g, 0.76 mmol), N,N-diisopropylethylamine (140 μL, 0.82 mmol), and BOP (0.34 g, 0.76 mmol) were mixed and stirred in 3 mL of dehydrated acetonitrile. Next, the powdery PluPRβCD3 (0.5 g, 0.02 mmol) was gradually added to the mixture, and the whole was stirred at 25° C. for 48 hours. After the reaction, the reaction liquid was dropped into diethyl ether, and the resultant precipitate was washed with acetonitrile and centrifuged. The remaining white powder was dissolved in dichloromethane, and the solution was filtered. After that, the filtrate was dissolved in water, and the solution was dialyzed. Finally, the dialyzed product was freeze-dried to provide a PluPRβCD2 having tritylamide as a capping group.

δH (DMSO-d₆): 1.03-1.05 (br, 3H, Cⱼ—H), 3.28-3.38 (br, C_{b,d}—H and C_{k,l}—H), 3.42-3.66 (br, 32H, C_{c,e,f}—H, C_{m,n}—H), 4.45-4.47 (m, 7H, OH_{g}), 4.82-4.83 (m, 7H, Cₐ—H), 5.68-5.75 (m, 14H, OH_{h,i}), 7.16-7.32 (m, trityl group)

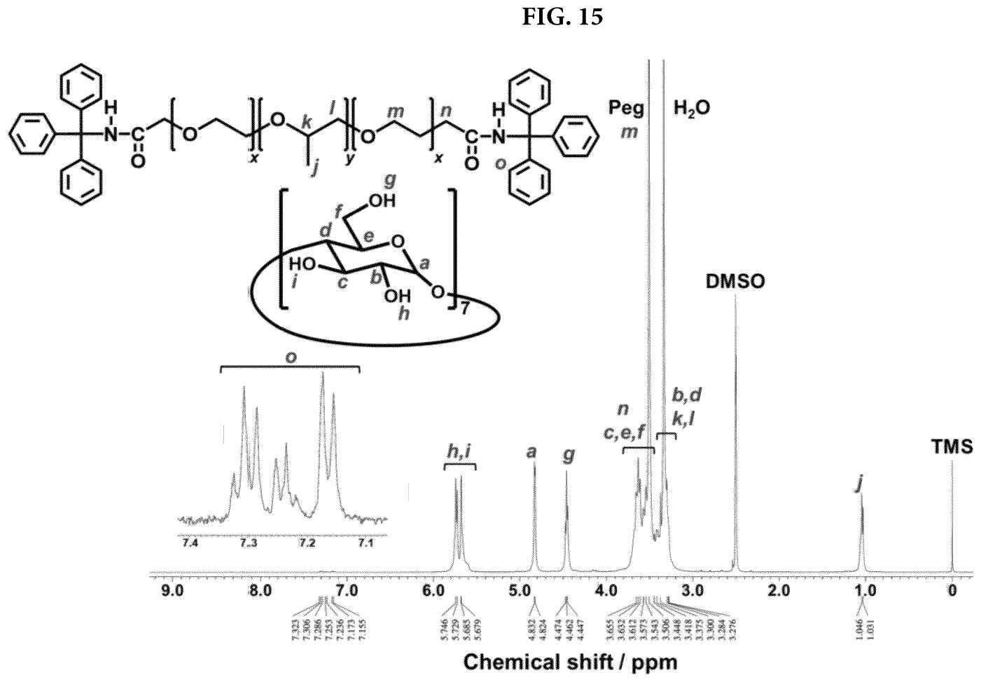

The synthesis of the PluPRβCD2 was recognized because a capping group-derived signal (o) was observed in the ¹H NMR spectrum of FIG. 15. In addition, the inclusion ratio of the polyrotaxane was calculated to be 6 mol % from an intensity ratio between the Pluronic-derived signal (j) and the β-cyclodextrin-derived signal (a).

A method of calculating the inclusion ratio is as described below.

The intensity of the signal (j) was 90H per one Pluronic chain, and hence the portion was normalized to 90H. At this time, the intensity of the β-cyclodextrin-derived signal (a) became 84H. It was calculated that 12 β-cyclodextrin molecules were present (84H/7H=12) because the intensity of the signal (a) per one β-cyclodextrin molecule was 7H. Meanwhile, the entirety of the Pluronic chain has 200 repeating units (85×2+30=200). The inclusion ratio was calculated to be (12/200)×100=6 mol % because the ratio was the ratio of the number of the β-cyclodextrin molecules to the number of the repeating units of the entirety of the Pluronic chain.

### (Introduction of Aldehyde Group (Synthesis of PluPRβCD1))

The PluPRβCD2 (120 mg, corresponding to 0.065 mmol of βCD), TEMPO (1.1 mg, 0.0065 mmol), and PhI(OAc)₂ (21 mg, 0.065 mmol) were dissolved in 3 mL of hexamethylphosphoric triamide, and the solution was stirred at 4° C. for 10 days. After the reaction, the solution was dropped into diethyl ether, and the precipitate was washed with acetonitrile. The resultant precipitate was dissolved in water, and the solution was dialyzed. Finally, the dialyzed product was freeze-dried to provide a PluPRβCD1 as white powder.

### (Quantitative Detection of Aldehyde Group)

The PluPRβCD1 (10 mg) was dissolved in an aqueous solution of 1,1-dimethylhydrazine (DMHZ) (0.2 M, pH=8.2), and the resultant solution was stirred at 37° C. for 48 hours. Next, NaBH₃CN was added to the solution so as to have a final concentration of 0.2 M, and the reaction liquid was further stirred at 37° C. for 48 hours. After the reaction, the reaction liquid was dialyzed with a dialysis membrane having a MWCO of 1 kDa for 24 hours, and was freeze-dried. Finally, the resultant powder was washed with ethanol, and was dried under reduced pressure to provide a PluPRβCD1 having an aldehyde group labeled with DMHZ.

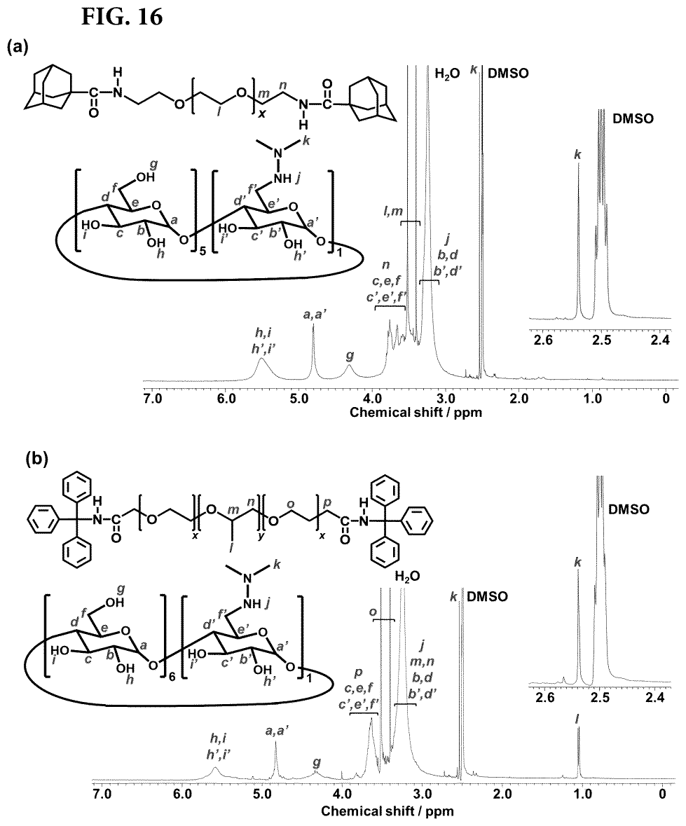

Assignment of ¹H NMR Spectrum PluPRβCD1-DMHZ δH (DMSO-d₆): 1.04-1.05 (br, 3H, Cₗ—H), 2.54 (s, 3H, Cₖ-H), 3.25 (br, Nⱼ—H, C_{b,b′,c,c′}—H and C_{m,n}—H), 3.40-3.52 (m, 4H, Co—H), 3.64-3.82 (br, 32H, Cₚ—H, C_{c,e,f}—H and C_{c′,e′,f′}—H), 4.34 (br, 7H, OH_{g}), 4.83 (br, 7H, C_{a,a′}—H), 5.59 (br, 14H, OH_{h,h′,i,i′}) (FIG. 16b)

With regard to FIG. 16b, a broad spectrum along with the formation of the polyrotaxane was observed. In particular, the spectrum was significantly observed in each of signals (g) and (h,h′,i,i′). The introduction ratio of the polyrotaxane was calculated to be 6 mol % from a clear signal (1) derived from an axile polymer and a β-cyclodextrin-derived signal (a,a′). In addition, the labeling of an aldehyde group with DMHZ resulted in the appearance of a DMHZ-derived signal (k). It was recognized from an intensity ratio between the signals (a,a′) and (k) that one aldehyde group was introduced per one β-cyclodextrin molecule.

Method of calculating inclusion ratio: the intensity of the signal (j) was 90H per one Pluronic chain, and hence the portion was normalized to 90H. At this time, the intensity of the β-cyclodextrin-derived signal (a,a′) became 84H. It was calculated that 12 β-cyclodextrin molecules were present (84H/7H=12) because the intensity of the signal (a,a′) per one 3-cyclodextrin molecule was 7H. Meanwhile, the entirety of the Pluronic chain has 200 repeating units (85×2+30=200). The inclusion ratio was calculated to be (12/200)×100=6 mol % because the ratio was the ratio of the number of the β-cyclodextrin molecules to the number of the repeating units of the entirety of the Pluronic chain. In addition, when the signal (a,a′) was normalized to 7H, the intensity of the DMHZ-derived signal (k) became δH. The intensity of the signal (k) per one molecule is δH, and hence one DMHZ molecule is present per one β-cyclodextrin molecule. In the end, it was concluded that one aldehyde group was present per one β-cyclodextrin molecule because the equivalent of DMHZ and the equivalent of an aldehyde group were equal to each other.

### Example 8

In this Example, inclusion ratio-controlled polyrotaxanes (a Peg₂₀ₖPRαCD1 and a Peg₁₀ₖPRαCD1) of the present invention were synthesized.

The synthesis method of Example 5 was adopted except that the constituent unit for a linear molecule was changed to Peg₁₀ₖ-OH or Peg₂₀ₖ-OH. Peg₁₀ₖ-OH and Peg₂₀ₖ-OH were purchased from FUJIFILM Wako Pure Chemical Corporation.

### (Quantitative Detection of Aldehyde Group)

The PegPRαCD1 was dissolved in an aqueous solution of DMHZ (0.2 M, pH=8.2), and the resultant solution was stirred at 37° C. for 48 hours. Next, NaBH₃CN was added to the solution so as to have a final concentration of 0.2 M, and the reaction liquid was further stirred at 37° C. for 48 hours. After the reaction, the reaction liquid was dialyzed with a dialysis membrane having a MWCO of 1 kDa for 24 hours, and was freeze-dried. Finally, the resultant powder was washed with ethanol, and was dried under reduced pressure to provide a PegPRαCD1 having an aldehyde group labeled with DMHZ.

### Assignment of ¹H NMR Spectrum

PegPRαCD1-DMHZ δH (DMSO-d₆): 2.54 (s, 3H, Cₖ—H), 3.24 (br, Nⱼ—H, C_{b,b′,d,d′}—H), 3.40-3.52 (m, 4H, C_{l,m}—H), 3.59-3.81 (br, 28H, Cₙ—H, C_{c,e,f}—H and C_{c′,e′,f′}—H), 4.31 (br, δH, OH_{g}), 4.80-4.81 (br, δH, C_{a,a′}—H), 5.51 (br, δH, OHh,h′,i,i′) (FIG. 16a)

With regard to FIG. 16a, a broad spectrum along with the formation of the polyrotaxane was observed. In particular, the spectrum was significantly observed in each of signals (g) and (h,h′,i,i′). The introduction ratio of the polyrotaxane was calculated to be 20 mol % from an axile polymer-derived signal (l,m) and an α-cyclodextrin-derived signal (n,c,e,f,c′,e′,f′). In addition, the labeling of an aldehyde group with DMHZ resulted in the appearance of a DMHZ-derived signal (k). It was recognized from an intensity ratio between the signals (a,a′) and (k) that one aldehyde group was introduced per one α-cyclodextrin molecule.

Method of calculating inclusion ratio: the α-cyclodextrin-derived signal (a,a′) was normalized to δH that was an intensity per one molecule. At this time, the intensity of the polyethylene glycol-derived signal (l,m) became 20H. 20H corresponds to five repeating units because the intensity of the polyethylene glycol per repeating unit is 4H. That is, the inclusion ratio of the polyrotaxane was calculated to be (1/5)×100=20 mol % because one α-cyclodextrin molecule was present per five repeating units of the polyethylene glycol. In addition, when the signal (a,a′) was normalized to δH, the intensity of the DMHZ-derived signal (k) became δH. The intensity of the signal (k) per one molecule was δH, and hence it was calculated that one DMHZ molecule was present per one α-cyclodextrin molecule. In the end, it was concluded that one aldehyde group was present per one α-cyclodextrin molecule because the equivalent of DMHZ and the equivalent of an aldehyde group were equal to each other.

### Example 9

### (Production, and Identification of Mechanical Properties, 2 of Thread-Like Atelocollagen of the Present Invention)

The production of thread-like atelocollagen of the present invention, and the identification of the mechanical properties thereof were performed in the same manner as in Examples described above. To put it simply, a solution (25 mg/mL) of atelocollagen in a 0.1 M phosphate buffer (pH=7.0) was poured into a 0.05 M phosphate buffer at 37° C. through an 18 G plastic tube. The resultant atelocollagen thread was crosslinked to the Peg₂₀ₖPRαCD1, the Peg₁₀ₖPRαCD1, the Plu₁₅ₖβCD1, or the Plu₉ₖβCD1 in a 0.1 M borate buffer (pH=8.5) via stepwise reductive amination. The crosslinked thread was washed with an aqueous solution of ethanol, and was dried at ambient temperature. Finally, the thread was fixed to a flexible polypropylene sheet for a tensile test. The fixed sample was immersed in a 50 mM phosphate buffer (pH=7) for 3 minutes, and was then subjected to a tensile test under a wet state. SHIMADZU MICRO AUTOGRAPH MST-X HS/HR was used in the tensile test.

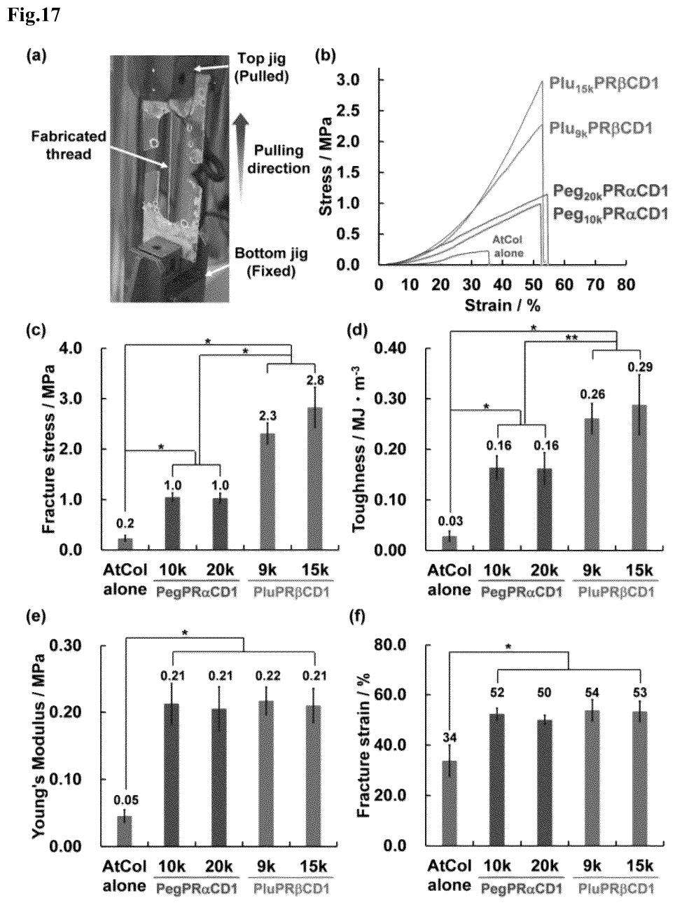

The results of the tensile test are shown in FIG. 17 (FIG. 17a is a typical photograph during the performance of the tensile test). The crosslinking with each of the Peg₁₀ₖPRαCD1 and the Peg₂₀ₖPRαCD1 significantly increased the fracture stress and fracture elongation percentage of the atelocollagen thread (AtCol) (FIG. 17b, FIG. 17c, and FIG. 17f). Along with the increases, the toughness thereof increased by a factor of 5 (FIG. 17d). At the initial stage of the stress-strain curve thereof, the stress and elongation percentage thereof were in a linear relationship, and hence the Young's modulus thereof was calculated from the region. As a result, the crosslinking with each of the Peg₁₀ₖPRαCD1 and the Peg₂₀ₖPRαCD1 increased the Young's modulus by a factor of 4 (FIG. 17e). Meanwhile, even when the molecular weight of a Peg chain was changed, no significant changes in mechanical properties of the thread-like atelocollagen were observed.

The crosslinking with each of the Plu₁₉ₖPRβCD1 and the Plu₁₅ₖPRβCD1 further improved the mechanical properties of the thread-like atelocollagen. That is, the fracture stress thereof further increased by a factor of 2.3 as compared to those in the cases of the PegPRαCD1s (FIG. 17b and FIG. 17c). Along with the increase, the toughness thereof further increased by a factor of 1.6 (in each of FIG. 17c and FIG. 17d, no statistically significant difference was present between the Plu₉ₖPRβCD1 and the Plu₁₅ₖPRβCD1). Meanwhile, the Young's modulus and fracture elongation percentage thereof did not change (FIG. 17e and FIG. 17f). In addition, those results coincided with the results obtained by the crosslinking with the PegPRαCD1s in that the molecular weight of an axile polymer did not affect the mechanical properties of the thread-like atelocollagen.

That is, it was recognized that a reduction in inclusion ratio of cyclic molecules was more important for the toughening of the thread-like atelocollagen of the present invention than an increase in molecular weight of its PR (in particular, its linear molecule) was.

It is conceivable from the results of Table 1 and FIG. 17 above that a biomaterial having stretchability (in particular, collagen having stretchability) has one or more of the following mechanical properties:
(1) a fracture stress of from 280 kPa to 3,200 kPa;
(2) a fracture strain of from 40% to 70%;
(3) an elastic modulus of from 18 kPa to 220 kPa;
(4) a toughness of from 83 kJ/m³ to 350 kJ/m³; and
(5) a stress of from 10 kPa to 1,000 kPa at the time of the loading of a strain of from 30% to 40%.

### GENERAL REMARKS

To develop a biomaterial having stretchability, a novel aldehyde group-containing polyrotaxane was synthesized, and the biomaterial was crosslinked by reductive amination.

The use of a redox couple formed of a catalytic oxidizing agent 2,2,6,6-tetramethylpiperidine 1-oxyl (TEMPO) and a reducing agent (bis(trifluoroacetoxy)iodo)benzene (PhI(OAcTf)₂) enabled selective production of an aldehyde group in each of cyclodextrins (βCD and αCD) in the PRβCD1 and the PRαCD1.

As a result of a comparative investigation performed with Dess-Martin periodinane, it was recognized that the redox couple was suitable for the production of an aldehyde group in each of βCD and the PRβCD1.

As is apparent from the stress-strain curve and the experiment in which the strain loading was repeated, the crosslinking of the biomaterial with each of the PRβCD1 and the PRαCD1 resulted in significant improvements in mechanical strength and softness of the biomaterial. Further, not only the stretchability of the biomaterial but also the airframe property thereof can be controlled by changing the kinds of its linear molecule, capping groups (stopper molecules), and cyclic molecules.

The control of the inclusion ratio of the cyclic molecules can achieve the toughening of the biomaterial.

### INDUSTRIAL APPLICABILITY

There can be provided an aldehyde group-added cyclic molecule-containing polyrotaxane and a biomaterial having stretchability (in particular, collagen having stretchability).

1-13. (canceled)

14. A polyrotaxane, the polyrotaxane comprising the following:
a linear molecule;
capping groups (stopper molecules), provided that the capping groups are positioned at both terminals of the linear molecule; and
an aldehyde group-added cyclic molecule, provided that an inside of the cyclic molecule is penetrated by the linear molecule, and
wherein the aldehyde group-added cyclic molecule is substantially free of a ketone group added thereto.

15. A biomaterial subjected to crosslinking treatment with the polyrotaxane according to claim 14.

16. The biomaterial according to claim 14, wherein the biomaterial is collagen.

17. The biomaterial according to claim 15, wherein the biomaterial is thread-like collagen.

18. The biomaterial according to claim 17, wherein the linear molecule is a constituent unit based on polyethylene glycol, and the aldehyde group-added cyclic molecule is a constituent unit based on an aldehyde group-added cyclodextrin.

19. The biomaterial according to claim 17, wherein the linear molecule is a constituent unit based on polypropylene glycol and the aldehyde group-added cyclic molecule is a constituent unit based on an aldehyde group-added cyclodextrin.

20. The biomaterial according to claim 17, wherein the linear molecule is a constituent unit based on poly(ethylene glycol)-block-poly(propylene glycol)-block-poly(ethylene glycol) and the aldehyde group-added cyclic molecule is a constituent unit based on an aldehyde group-added cyclodextrin.

21. The biomaterial according to claim 18, wherein the biomaterial has the following properties:
(1) a fracture stress of from 280 kPa to 3,200 kPa;
(2) a fracture strain of from 40% to 70%;
(3) an elastic modulus of from 18 kPa to 220 kPa; and
(4) a toughness of from 83 kJ/m³ to 350 kJ/m³.

22. The biomaterial according to claim 19, wherein the biomaterial has the following properties:
(1) a fracture stress of from 280 kPa to 3,200 kPa;
(2) a fracture strain of from 40% to 70%;
(3) an elastic modulus of from 18 kPa to 220 kPa; and
(4) a toughness of from 83 kJ/m³ to 350 kJ/m³.

23. The biomaterial according to claim 20, wherein the biomaterial has the following properties:
(1) a fracture stress of from 280 kPa to 3,200 kPa;
(2) a fracture strain of from 40% to 70%;
(3) an elastic modulus of from 18 kPa to 220 kPa; and
(4) a toughness of from 83 kJ/m³ to 350 kJ/m³.

24. The biomaterial according to claim 21, wherein the biomaterial further has the following property:
(1) a stress of from 10 kPa to 1,000 kPa at a time of loading of a strain of from 30% to 40%.

25. A biomaterial subjected to crosslinking treatment with a polyrotaxane, the polyrotaxane comprising the following:
a linear molecule;
capping groups (stopper molecules), provided that the capping groups are positioned at both terminals of the linear molecule;
an aldehyde group-added cyclic molecule, provided that an inside of the cyclic molecule is penetrated by the linear molecule and
wherein a fracture strain of the biomaterial is from 40% to 70%.

26. The biomaterial according to claim 25, wherein the biomaterial has the following properties:
(1) a fracture stress of from 280 kPa to 3,200 kPa;
(2) an elastic modulus of from 18 kPa to 220 kPa; and
(3) a toughness of from 83 kJ/m³ to 350 kJ/m³.

27. The biomaterial according to claim 26, wherein the linear molecule is a constituent unit based on polyethylene glycol, an aldehyde group-added cyclic molecule is a constituent unit based on an aldehyde group-added cyclodextrin, and the biomaterial is thread-like collagen.

28. The biomaterial according to claim 26, wherein the linear molecule is a constituent unit based on polypropylene glycol, an aldehyde group-added cyclic molecule is a constituent unit based on an aldehyde group-added cyclodextrin, and the biomaterial is thread-like collagen.

29. The biomaterial according to claim 26, wherein the linear molecule is a constituent unit based on poly(ethylene glycol)-block-poly(propylene glycol)-block-poly(ethylene glycol), an aldehyde group-added cyclic molecule is a constituent unit based on an aldehyde group-added cyclodextrin, and the biomaterial is thread-like collagen.

30. A method of producing a biomaterial subjected to crosslinking treatment with a polyrotaxane, the method comprising the following step:
(1) a step of subjecting a biomaterial having a lysine residue to reductive amination reaction treatment in the presence of an aldehyde group-added cyclic molecule-containing polyrotaxane.

31. The production method according to claim 30, wherein the step (1) is a step of bringing the biomaterial having a lysine residue into contact with a buffer containing the aldehyde group-added cyclic molecule-containing polyrotaxane to subject the biomaterial having a lysine residue to the reductive amination reaction treatment.

32. The production method according to claim 31, wherein the buffer in the step (1) contains a hydride reducing agent.

33. The production method according to claim 32, wherein the biomaterial is thread-like collagen.
